SEQUENCE LISTING (1 sequence)

SEQ ID NO: 1 - protein, 23 aa, Homo sapiens
KKKVSRSGLYRSPSMPENLNRPR

(19) United States
(12) Patent Application Publication
(10) Pub. No.: US 2024/0252544 A1
(43) Pub. Date: Aug. 1, 2024

(54) Method for Producing Retinal Tissue

(71) Applicants: RIKEN, Saitama (JP); Sumitomo Pharma Co., Ltd., Osaka-shi, Osaka (JP)

(72) Inventors: Michiko Mandai, Wako-shi, Saitama (JP); Suguru Yamasaki, Chuo-ku, Kobe-shi, Hyogo (JP)

(73) Assignees: RIKEN, Saitama (JP); Sumitomo Pharma Co., Ltd., Osaka-shi, Osaka (JP)

(21) Appl. No.: 18/290,392

(22) PCT Filed: May 13, 2022
(86) PCT No.: PCT/JP2022/020264

§ 371 Date: Nov. 13, 2023

(30) Foreign Application Priority Data
May 14, 2021 (JP) 2021-082246

(51) Int. Cl. A61K 35/30; C12N 5/079

(52) U.S. Cl. CPC A61K 35/30; C12N 5/0618; C12N 2501/155; C12N 2501/727; C12N 2506/02; C12N 2513/00; C12N 2533/52

(57) ABSTRACT

An object of the present invention is to provide a method for producing a retinal cell or retinal tissue with a reduced proportion of non-target cells in differentiating pluripotent stem cells. The method of the present invention for producing a retinal cell or retinal tissue comprises: (A) suspension-culturing a pluripotent stem cell to form a cell aggregate of pluripotent stem cells; and (B) suspension-culturing the cell aggregate obtained in the step (A) in the presence of a BMP signaling pathway agonist and a CHK1 signaling pathway inhibitor to obtain a cell aggregate containing a retinal cell.

### TECHNICAL FIELD

The present invention relates to a method for producing a retinal cell or retinal tissue. The present invention also relates to a sphere-like cell aggregate produced by the production method, the sphere-like cell aggregate comprising a multilayer structure of an outer structure including a retinal pigment epithelial cell and an inner structure including a neural retina.

### BACKGROUND ART

It has recently become possible to produce retinal tissue by differentiating pluripotent stem cells. For example, a method for producing multilayered retinal tissue from a pluripotent stem cell (Patent Literature 1 and Non Patent Literature 1) has been reported. In addition, a method of obtaining multilayered retinal tissue by forming a homogeneous aggregate of pluripotent stem cells in a serum-free medium containing a Wnt signaling pathway inhibitor, suspension-culturing the aggregate in the presence of a basal membrane preparation, and then suspension-culturing the aggregate in a serum medium (Patent Literature 2 and Non Patent Literature 2), a method of obtaining multilayered retinal tissue by forming a homogeneous aggregate of pluripotent stem cells in a culture medium containing a BMP4 signaling pathway activator (Patent Literature 3 and Non Patent Literature 3), and a method of obtaining multilayered retinal tissue by naturally differentiating adhered pluripotent stem cells and separating retinal tissue contained in part of them (Non Patent Literature 4) are known.

However, these production methods suffer from a certain level of variation in the quality of retinal tissue, and non-target cells may be induced in a certain proportion. In using such retinal tissue as transplant tissue, in particular, strict management of the quality is demanded.

Meanwhile, PD407824 (CHK1: checkpoint kinase 1 inhibitor) is known as a sensitizer for BMP. Use of PD407824 and BMP in combination has been reported to enhance the differentiation of ES cells into mesodermal cells (Non Patent Literature 5). However, an effect of PD407824 on the nervous system including retinal tissue has not yet been known.

### CITATION LIST

### Patent Literature

Patent Literature 1: WO2011/055855
Patent Literature 2: WO2013/077425
Patent Literature 3: WO2015/025967

### Non Patent Literature

Non Patent Literature 1: Eiraku M. et al., “Self-organizing optic-cup morphogenesis in three-dimensional culture”, Nature, 472, 51-56, (2011)
Non Patent Literature 2: Nakano T. et al., “Self-formation of Optic Cups and Storable Stratified Neural Retina From Human ESCs” Cell Stem Cell, 10(6), 771-785, (2012)
Non Patent Literature 3: Kuwahara A. et al., “Generation of a ciliary margin-like stem cell niche from self-organizing human retinal tissue” Nature Communications, 6, Article number: 6286, (2015)
Non Patent Literature 4: Xiufeng Zhong et al., “Generation of three-dimensional retinal tissue with functional photoreceptors from human iPSCs”, Nature Communications volume 5, Article number: 4047 (2014)
Non Patent Literature 5: Lingling Feng et al., “Discovery of a Small-Molecule BMP Sensitizer for Human Embryonic Stem Cell Differentiation”, Cell Reports, Volume 15, Issue 9, 31 May 2016, Pages 2063-2075

### SUMMARY OF INVENTION

### Technical Problem

An object of the present invention is, in view of the above circumstances, to provide a method for producing a retinal cell or retinal tissue with a reduced proportion of non-target cells in differentiating pluripotent stem cells and thus improved quality.

### Solution to Problem

The present inventors have found that, in a method for producing retinal tissue with BMP, the amount of BMP to be added can be reduced to about 1/10 of a typical concentration by using a CHK1 signaling pathway inhibitor in combination. In addition, the present inventors have found that retinal tissue of predetermined shape with a reduce proportion of non-target cells compared with that in the case of using BMP alone can be produced by combining BMP and a CHK1 signaling pathway inhibitor. Furthermore, the present inventors have found that a sphere-like cell aggregate comprising a multilayer structure in which a retinal pigment epithelium (RPE) is localized in the outside and retinal tissue (neural retinal tissue) is localized in the inside can be selectively produced by using typical concentrations of BMP and a CHK1 signaling pathway inhibitor in combination.

Specifically, the present invention relates to each of the following aspects.

[1]

A method for producing a retinal cell or retinal tissue, comprising:
(A) suspension-culturing a pluripotent stem cell to form a cell aggregate of pluripotent stem cells; and
(B) suspension-culturing the cell aggregate obtained in the step (A) in the presence of a BMP signaling pathway agonist and a CHK1 signaling pathway inhibitor to obtain a cell aggregate containing a retinal cell.
   [2]

The production method according to [1], wherein, in the step (B), the BMP signaling pathway agonist is present in such a concentration that an almost truly spherical cell aggregate is formed and differentiation into a retinal pigment epithelial cell is suppressed.

[3]

The production production method according to [1], wherein the cell aggregate obtained in the step (B) is a sphere-like cell aggregate comprising a multilayer structure of an outer structure including a retinal pigment epithelial cell and an inner structure including a neural retina, and, in the step (B), the BMP signaling pathway agonist is present in such a concentration that the sphere-like cell aggregate comprising a multilayer structure is formed.

[4]

The production method according to any one of [1] to [3], wherein the CHK1 signaling pathway inhibitor is PD407824.

[5]

The production method according to any one of [1] to [4], wherein the BMP signaling pathway agonist is one or more proteins selected from the group consisting of BMP2, BMP4, BMP7 and GDF7.

[6]

The production method for producing a retinal cell or retinal tissue according to any one of [1] to [5], wherein, in the step (B), the BMP signaling pathway agonist is added to a culture medium between Day 2 and Day 9 after start of suspension culture in the step (A).

[7]

The production method according to any one of [1] to [6], wherein, in the step (B), the CHK1 signaling pathway inhibitor is added to a culture medium simultaneously with the BMP signaling pathway agonist.

[8]

The production method according to any one of [1] to [7], wherein a concentration of the CHK1 signaling pathway inhibitor is such a concentration that a CHK1 signaling pathway inhibitory effect comparable to a CHK1 signaling pathway inhibitory effect of 0.1 μM to 10 μM PD407824 is exerted.

[9]

The production method according to any one of [1] to [8], further comprising dissecting retinal tissue of required size for transplantation from the cell aggregate obtained in the step (B).

[10]

A sphere-like cell aggregate comprising:
a multilayer structure of an outer structure including a retinal pigment epithelial cell and an inner structure including a neural retina, wherein
(1) a neural retinal layer including at least a photoreceptor layer is formed in the neural retina in the inner structure, and the photoreceptor layer includes one or more cells selected from the group consisting of a photoreceptor cell, a photoreceptor progenitor cell and a retinal progenitor cell,
(2) the neural retina is present in a folded state in the inner structure,
(3) the retinal pigment epithelial cell in the outer structure is an RPE65-positive cell, an MITF-positive cell, or an RPE65-positive and MITF-positive cell, and
(4) the cell aggregate is free of a lens, a vitreous body, a cornea and a blood vessel.
   [11]

The sphere-like cell aggregate according to [10], wherein, in at least a part of the sphere-like cell aggregate, a basal surface of the retinal pigment epithelial cell is facing the inner structure, and a basal surface of the neural retina is facing the outer structure.

[12]

The sphere-like cell aggregate according to [10] or [11], wherein the retinal pigment epithelial cell and the neural retina are further connecting to each other as an epithelium structure, and the sphere-like cell aggregate further includes a ciliary marginal zone-like structure between the retinal pigment epithelial cell and the neural retina.

[13]

The sphere-like cell aggregate according to [12], wherein the ciliary marginal zone-like structure includes an Rdh10-positive cell, an Otx1-positive cell, and/or a Zic1-positive cell.

[14]

The sphere-like cell aggregate according to any one of [10] to [13], wherein 30% or more of the inner structure constitutes a neural retina.

[15]

The sphere-like cell aggregate according to any one of [10] to [14], having a diameter of 0.2 mm to 2 mm.

[16]

A method for producing the sphere-like cell aggregate according to any one of [10] to [15], comprising:
(A) suspension-culturing a pluripotent stem cell to form a cell aggregate of pluripotent stem cells; and
(B) suspension-culturing the cell aggregate obtained in the step (A) in the presence of a BMP signaling pathway agonist and a CHK1 signaling pathway inhibitor to obtain a sphere-like cell aggregate comprising a multilayer structure of an outer structure including a retinal pigment epithelial cell and an inner structure including a neural retina.
   [17]

A pharmaceutical composition (a composition for transplantation, transplant tissue or a transplant) comprising:
the sphere-like cell aggregate according to any one of [10] to [15] or a part thereof.
   [18]

A method for treating a disease caused by damage of a retinal cell or retinal tissue or injury of retinal tissue, comprising:
transplanting the sphere-like cell aggregate according to any one of [10] to [15] or a part thereof to a subject in need of transplantation.
   [19]

Use of the sphere-like cell aggregate according to any one of [10] to [15] or a part thereof in production of a pharmaceutical composition for treating a disease caused by damage of a retinal cell or retinal tissue or injury of retinal tissue.

Use of the sphere-like cell aggregate according to any one of [10] to [15] or a part thereof in treating a disease caused by damage of a retinal cell or retinal tissue or injury of retinal tissue.

### Advantageous Effects of Invention

According to the present invention, a method for producing a retinal cell or retinal tissue with a reduced proportion of non-target cells compared with that in the case of using BMP alone can be provided by combining BMP and PD407824, and retinal tissue of favorable shape can be produced.

### BRIEF DESCRIPTION OF DRAWINGS

FIG. 1 is fluorescence microscope images of observation of the states of aggregated masses on Day 17 from initiation of suspension culture with addition of 1 μM PD407824 (“PD” in the figure) together with BMP4 (0.15 nM or 1.5 nM) in Example 1.

FIG. 2 is fluorescence microscope images of observation of the states of aggregated masses on Day 15 from initiation of suspension culture with addition of 1.5 nM BMP4 alone, or 0.15 nM BMP4 and 1 μM PD407824 (“PD” in the figure) in Example 2.

FIG. 3 is fluorescence microscope images of observation of the states of aggregated masses on Day 15 from initiation of suspension culture with addition of 1.5 nM BMP4 alone, or 0.15 nM BMP4 and 1 μM PD407824 (“PD” in the figure) in Example 2.

FIG. 4 is bright field microscope images and fluorescence microscope images of observation of the states of aggregated masses on Day 36 from initiation of suspension culture with addition of 1.5 nM BMP4 alone, or 0.15 nM BMP4 and 1 μM PD407824 (“PD” in the figure) in Example 2.

FIG. 5 is bright field microscope images and fluorescence microscope images of observation of the states of aggregated masses on Day 21 from initiation of suspension culture with addition of 1 μM PD407824 (“PD” in the figure) together with 1.5 nM BMP4 in Example 3.

FIG. 6 is photographs of observation through a confocal fluorescence microscope for immunostained sections of aggregated masses on Day 25 from initiation of suspension culture with addition of 1 μM PD407824 (“PD” in the figure) together with 1.5 nM BMP4 in Example 3.

FIG. 7 is photographs of observation through a confocal fluorescence microscope for immunostained sections of aggregates on Day 25 from initiation of suspension culture with addition of 1 μM PD407824 together with 1.5 nM BMP4 in Example 3.

FIG. 8 is fluorescence microscope images of observation of the states of aggregates on Day 9 after the start of suspension culture with addition of different concentrations of PD407824 together with different concentrations of BMP4 in Example 4.

FIG. 9 is photographs of observation through a confocal fluorescence microscope for immunostained sections of aggregates on Day 60 from initiation of suspension culture with addition of 1 μM PD407824 together with 1.5 nM BMP4 in Example 5.

### DESCRIPTION OF EMBODIMENTS

### Definition

The “stem cells” refer to undifferentiated cells having differentiation potency and proliferation potency (particularly, self-renewal ability). In the stem cells, subgroups of pluripotent stem cells, multipotent stem cells and unipotent stem cells, are included according to the differentiation potency. The pluripotent stem cells refer to stem cells that can be cultured in vitro and has an ability (pluripotency) to be able to differentiate into three germ layers (ectoderm, mesoderm, endoderm) and/or all cell lineages belonging to the extraembryonic tissue. The multipotent stem cells refer to stem cells having an ability to differentiate into a plurality of tissues or cells, although the definition is not applied to all of them. The unipotent stem cells refer to stem cells having an ability to be able to differentiate into a predetermined tissue or cells.

The “pluripotent stem cells” can be induced from, e.g., a fertilized egg, a cloned embryo, germline stem cells, tissue stem cells and somatic cells. Examples of the pluripotent stem cells can include embryonic stem cells (ES cells), embryonic germ cells (EG cells) and induced pluripotent stem cells (iPS cells). Muse cells (Multi-lineage differentiating stress enduring cells) obtained from the mesenchymal stem cells (MSC) and GS cells prepared from germ cells (for example, testis) are included in the pluripotent stem cells. Human embryonic stem cells are those established from the human embryo within 14 days after fertilization.

Human embryonic stem cells were established in 1998 and have been used also for regenerative medicine. The embryonic stem cells can be produced by culturing inner cell aggregate on feeder cells or a culture medium containing bFGF. The method for producing embryonic stem cells is described, for example, in WO96/22362, WO02/101057, U.S. Pat. Nos. 5,843,780, 6,200,806, 6,280,718. The embryonic stem cells are available from a predetermined institution and also, commercially available. For example, human embryonic stem cells such as KhES-1, KhES-2 and KhES-3 are available from the Institute for Frontier Life and Medical Sciences, Kyoto University. Human embryonic stem cells such as human ES cells genetically engineered so as to have Rx::Venus, Rx::AcGFP and Crx::Venus reporter genes (KhES-1 strain (Non Patent Literature 3)) are available from RIKEN.

The “induced pluripotent stem cells” refers to cells having pluripotency, which is induced by reprogramming somatic cells by a method known in the art.

The induced pluripotent stem cells were established in mouse cells by Yamanaka et al., in 2006 (Cell, 2006, 126 (4), pp. 663-676). The induced pluripotent stem cells were also established in human fibroblasts in 2007. The induced pluripotent stem cells have pluripotency and self-renewal ability similarly to embryonic stem cells (Cell, 2007, 131 (5), pp. 861-872; Science, 2007, 318 (5858), pp. 1917-1920; Nat. Biotechnol., 2008, 26 (1), pp. 101-106).

The induced pluripotent stem cells more specifically refer to cells which are induced to be pluripotent by reprogramming somatic cells differentiated into, for example, fibroblasts and peripheral blood mononuclear cells, by allowing any one of sets of a plurality of genes selected from a reprogramming gene group containing Oct3/4, Sox2, Klf4, Myc (c-Myc, N-Myc, L-Myc), Glis1, Nanog, Sall4, lin28 and Esrrb to express. Examples of a preferable set of reprogramming factors may include (1) Oct3/4, Sox2, Klf4, and Myc (c-Myc or L-Myc) and (2) Oct3/4, Sox2, Klf4, Lin28 and L-Myc (Stem Cells, 2013; 31: 458-466).

Other than producing induced pluripotent stem cells through direct reprogramming by gene expression, the pluripotent stem cells can be artificially induced from somatic cells, for example, by adding a chemical compound (Science, 2013, 341, pp. 651-654).

Alternatively, an induced pluripotent stem cell strain is available. For example, human induced pluripotent stem cell strains established by Kyoto University, such as 201B7 cell, 201B7-Ff cell, 253G1 cell, 253G4 cell, 1201C1 cell, 1205D1 cell, 1210B2 cell and 1231A3 cell, are available form Kyoto University and iPS Academia Japan, Inc. As the induced pluripotent stem cells, for example, Ff-101 cell, Ff-114 cell and QHJI01s04 cell established by Kyoto University, are available from Kyoto University.

In the specification, the pluripotent stem cells are preferably embryonic stem cells or induced pluripotent stem cells, more preferably induced pluripotent stem cells.

In the specification, the pluripotent stem cells are human pluripotent stem cells, preferably human induced pluripotent stem cells (iPS cells) or human embryonic stem cells (ES cells).

Pluripotent stem cells such as human iPS cells can be subjected to maintenance culture and expansion culture performed by methods known to those skilled in the art.

The “retinal tissue” means a tissue in which a single type or a plurality of types of retinal cells constituting each retinal layer in a retina in vivo are present according to a predetermined order. The “neural retina (NR)” is a retinal tissue and means a tissue containing an inside neural retinal layer that does not contain a retinal pigment epithelial layer among retinal layers mentioned later.

The “retinal cells” mean cells constituting each retinal layer in a retina in vivo or progenitor cells thereof. In the retinal cells, cells such as photoreceptor cells (rod photoreceptor cell, cone photoreceptor cell), horizontal cells, amacrine cells, intermediate neuronal cells, retinal ganglion cells (ganglion cell), bipolar cells (rod bipolar cell, cone bipolar cell), Muller glial cells, retinal pigment epithelial (RPE) cells, ciliary body, their progenitor cells (e.g., photoreceptor progenitor cell, bipolar progenitor cell), and retinal progenitor cells are included, though not limited thereto. Among the retinal cells, examples of cells constituting a neural retinal layer (also referred to as neural retina cells or neural retina-related cells) specifically include cells such as photoreceptor cells (rod photoreceptor cell, cone photoreceptor cell), horizontal cells, amacrine cells, intermediate neuronal cells, retinal ganglion cells (ganglion cell), bipolar cells (rod bipolar cell, cone bipolar cell), Muller glial cells, and their progenitor cells (e.g., photoreceptor progenitor cell, bipolar progenitor cell). In other words, in the neural retina-related cells, neither retinal pigment epithelial cells nor ciliary body cells are included.

The “matured retinal cells” mean cells that may be contained in the retinal tissue of a human adult, and specifically mean differentiated cells such as photoreceptor cells (rod photoreceptor cell, cone photoreceptor cell), horizontal cells, amacrine cells, intermediate neuronal cells, retinal ganglion cells (ganglion cell), bipolar cells (rod bipolar cell, cone bipolar cell), Muller glial cells, retinal pigment epithelial (RPE) cells, and ciliary body cells. The “immature retinal cells” mean progenitor cells (e.g., photoreceptor progenitor cell, bipolar progenitor cell, retinal progenitor cell) destined for differentiation into matured retinal cells.

The photoreceptor progenitor cells, the horizontal progenitor cells, the bipolar progenitor cells, the amacrine progenitor cells, the retinal ganglion progenitor cells, the Muller glial progenitor cells, and the retinal pigment epithelial progenitor cells refer to progenitor cells destined for differentiation into photoreceptor cells, horizontal cells, bipolar cells, amacrine cells, retinal ganglion cells, Muller glial cells, and retinal pigment epithelial cells, respectively.

The “retinal progenitor cells” are progenitor cells capable of differentiating into any one of the immature retinal cells such as photoreceptor progenitor cells, horizontal progenitor cells, bipolar progenitor cells, amacrine progenitor cells, retinal ganglion progenitor cells, Muller glial cells, and retinal pigment epithelial progenitor cells, and refer to progenitor cells also capable of eventually differentiating into any one of the matured retinal cells such as photoreceptor cells, rod photoreceptor cells, cone photoreceptor cells, horizontal cells, bipolar cells, amacrine cells, retinal ganglion cells, and retinal pigment epithelial cells.

The “photoreceptor cells” are present in the photoreceptor layer of a retina in vivo and plays a role in absorbing light stimuli and converting them to electrical signals. The photoreceptor cells have two types, cones which function in the light and rods which function in the dark (referred to as cone photoreceptor cells and rod photoreceptor cells, respectively). Examples of the cone photoreceptor cells can include S cone photoreceptor cells which express S-opsin and receive blue light, L cone photoreceptor cells which express L-opsin and receive red light, and M cone photoreceptor cells which express M-opsin and receive green light. The photoreceptor cells are matured after differentiation from photoreceptor progenitor cells. Whether or not cells are photoreceptor cells or photoreceptor progenitor cells can be readily confirmed by those skilled in the art, for example, through the expression of cell markers (Crx and Blimp1 expressed in photoreceptor progenitor cells, recoverin expressed in photoreceptor cells, rhodopsin, S-opsin and M/L-opsin expressed in mature photoreceptor cells, etc.) mentioned later or the formation of an outer segment structure. In an embodiment, the photoreceptor progenitor cells are Crx-positive cells, and the photoreceptor cells are rhodopsin-, S-opsin- and M/L-opsin-positive cells. In an embodiment, the rod photoreceptor cells are NRL- and rhodopsin-positive cells. In an embodiment, the S cone photoreceptor cells are S-opsin-positive cells, the L cone photoreceptor cells are L-opsin-positive cells, and the M cone photoreceptor cells are M-opsin-positive cells.

The presence of neural retina-related cells can be confirmed from the presence or absence of expression of a neural retina-related cell-related gene (hereinafter, also referred to as “neural retina-related cell marker” or “neural retina marker”). The presence or absence of expression of the neural retina-related cell marker, or the ratio of neural retina-related cell marker-positive cells in a cell population or a tissue can be readily confirmed by those skilled in the art. Examples thereof include an approach using an antibody, an approach using nucleic acid primers, and an approach using sequencing reaction. As the approach using an antibody, the expression of a protein of the neural retina-related cell marker can be confirmed, for example, by dividing the number of predetermined neural retina-related cell marker-positive cells by the total number of cells in accordance with an approach such as flow cytometry or immunostaining using a commercially available antibody. As the approach using nucleic acid primers, the expression of RNA of the neural retina-related cell marker can be confirmed by, for example, PCR, semiquantitative PCR, or quantitative PCR (e.g., real-time PCR). As the approach using sequencing reaction, the expression of RNA of the neural retina-related cell marker can be confirmed using, for example, a nucleic acid sequencer (e.g., next-generation sequencer).

Examples of the neural retina-related cell marker include Rx (also referred to as Rax) and PAX6 expressed in retinal progenitor cells, Rx, PAX6 and Chx10 (also referred to as Vsx2) expressed in neural retinal progenitor cells, and Crx and Blimp1 expressed in photoreceptor progenitor cells. Examples thereof also include Chx10 strongly expressed in bipolar cells, PKCα, Goα, VSX1 and L7 expressed in bipolar cells, TuJ1 and Brn3 expressed in retinal ganglion cells, calretinin and HPC-1 expressed in amacrine cells, calbindin expressed in horizontal cells, recoverin expressed in photoreceptor cells and photoreceptor progenitor cells, rhodopsin expressed in rod cells, Nrl expressed in rod photoreceptor cells and rod photoreceptor progenitor cells, S-opsin and LM-opsin expressed in cone photoreceptor cells, RXR-γ expressed in cone cells, cone photoreceptor progenitor cells and ganglion cells, TRβ2, OTX2 and OC2 expressed in cone photoreceptor cells that appear at the early phase of differentiation among cone photoreceptor cells, or progenitor cells thereof, and Pax6 commonly expressed in horizontal cells, amacrine cells and ganglion cells.

The “positive cells” mean cells expressing a predetermined marker on the cell surfaces or within the cells. For example, the “Chx10-positive cells” mean cells expressing Chx10 protein.

The “retinal pigment epithelial cells” mean epithelial cells present outside the neural retina in a retina in vivo. Whether or not cells are retinal pigment epithelial cells can be readily confirmed by those skilled in the art, for example, through the expression of cell markers (MITF, Pax6, PMEL17, TYRP1, TRPM1, ALDH1A3, GPNMB, RPE65, CRALBP, MERTK, BEST1, TTR, etc.), the presence of melanin granules (brown-black), intercellular tight junctions, or polygonal/flagstone-like characteristic cell morphology. Whether or not cells have a function of retinal pigment epithelial cells can be readily confirmed, for example, from the ability to secrete cytokines such as VEGF and PEDF and the phagocytotic ability of photoreceptor outer segments. In an embodiment, the retinal pigment epithelial cells are RPE65-positive cells, MITF-positive cells, or RPE65-positive and MITF-positive cells.

The “retinal layer” means individual layers constituting the retina, and examples thereof can specifically include retinal pigment epithelial layer, photoreceptor layer, outer limiting membrane, outer nuclear layer, outer plexiform layer, inner nuclear layer, inner plexiform layer, ganglion cell layer, nerve fiber layer and inner limiting membrane.

The “neural retinal layer” means individual layers constituting the neural retina, and examples thereof can specifically include photoreceptor layer, outer limiting membrane, outer nuclear layer, outer plexiform layer, inner nuclear layer, inner plexiform layer, ganglion cell layer, nerve fiber layer and inner limiting membrane. The “photoreceptor layer” means a retinal layer that is formed in the outermost of the neural retina and is rich in a photoreceptor cell (rod photoreceptor cell, cone photoreceptor cell), a photoreceptor progenitor cell and a retinal progenitor cell. Each layer other than the photoreceptor layer is referred to as an inner layer. Which retinal layer the individual cells constitute can be confirmed by a known method, for example, by determining the presence or absence of expression or expression level of a cell marker.

In the case of retinal tissue at a stage where the appearance ratio of photoreceptor cells or photoreceptor progenitor cells is low, a layer containing proliferating neural retinal progenitor cells is referred to as “neuroblastic layer” and includes inner neuroblastic layer and outer neuroblastic layer. Those skilled in the art can make a judgment from the shade of color (the outer neuroblastic layer is light, and the inner neuroblastic layer is dark) by a known method, for example, under a bright field microscope.

The “ciliary body” includes “ciliary body” and “ciliary marginal zone” in the process of development and of an adult. Examples of a marker of the “ciliary body” include Zic1, MAL, HNF1beta, FoxQ1, CLDN2, CLDN1, GPR177, AQP1 and AQP4. Examples of the “ciliary marginal zone (CMZ)” can include a tissue that is present in a boundary region between the neural retina and the retinal pigment epithelium in a retina in vivo, and is a region containing tissue stem cells of the retina (retinal stem cells). The ciliary marginal zone is also called ciliarymargin or retinal margin, and the ciliary marginal zone, the ciliary margin and the retinal margin are equivalent tissues. The ciliary marginal zone is known to play an important role in the supply of retinal progenitor cells or differentiated cells to retinal tissue, the maintenance of a retinal tissue structure, etc. Examples of a marker gene of the ciliary marginal zone can include Rdh10 gene (positive), Otx1 gene (positive) and Zic1 (positive). The “ciliary marginal zone-like structure” is a structure similar to the ciliary marginal zone.

The “cell aggregate” is not particularly limited as long as a plurality of cells mutually adhere to form a three-dimensional structure, and refers to, for example, a mass formed by the aggregation of cells dispersed in a vehicle such as a culture medium, or a mass of cells formed through cell division. In the cell aggregate, the case of forming a predetermined tissue is also included.

The “sphere-like cell aggregate” means a cell aggregate having a stereoscopic shape close to a spherical shape. The stereoscopic shape close to a spherical shape is a shape having a three-dimensional structure, and examples thereof include a spherical shape that exhibits a circle or an ellipse when projected onto a two-dimensional surface, and a shape formed by overlapping a plurality of spherical shapes (e.g., which exhibits a shape formed by 2 to 4 circles or ellipses overlapping when two-dimensionally projected, and the shape is also referred to as “cloverleaf”). In an embodiment, the core part of the aggregate has a vesicular lamellar structure and is characterized in that the central part is observed to be dark and the outer edge portion is observed to be bright under a bright field microscope.

The “epithelial tissue” is a tissue formed by covering the body surface or the surface of a lumen (digestive tract, etc.), body cavity (pericardial cavity, etc.) or the like with cells without any space. The cells forming the epithelial tissue are referred to as epithelial cells. The epithelial cells have a polarity in the apical-basal direction. The epithelial cells can mutually and firmly join via adherence junction and/or tight junction to form a layer of the cells. A tissue formed from a single layer or dozen layers overlapping of this layer of the cells is the epithelial tissue. In a tissue capable of forming the epithelial tissue, retinal tissue, brain and spinal cord tissue, eyeball tissue, neural tissue or the like of a fetal stage and/or an adult is also included. In the specification, the neural retina is also the epithelial tissue. The “epithelial structure” means a structure characteristic of the epithelial tissue, such as apical surface or basal membrane.

The “continuous epithelial tissue” is a tissue having a continuous epithelium structure. The continuous epithelium structure is a structure where the epithelial tissue is continuously formed. The epithelium tissue continuously formed is a state in which 10 cells to 10⁷ cells, for example, in the tangent direction of the epithelial tissue, preferably 30 cells to 10⁷ cells, further preferably 10² cells to 10⁷ cells, in the tangent direction, are aligned.

For example, in the continuous epithelium structure formed in retinal tissue, the retinal tissue has an apical surface intrinsic to the epithelial tissue. The apical surface is formed almost in parallel to, for example, at least photoreceptor layer (outer nuclear layer) among the layers forming a neural retinal layer and continuously on the surface of the retinal tissue. For example, in the case of a cell aggregate containing retinal tissue prepared from pluripotent stem cells, the apical surface is formed on the surface of the aggregate, and 10 cells or more, preferably 30 cells or more, more preferably 100 cells or more, further preferably 400 cells or more of photoreceptor cells or photoreceptor progenitor cells are regularly and continuously arranged in the tangent direction of the surface.

In an embodiment, epithelial tissue is polarized so that “apical surface” and “basal membrane” are formed. The “basal membrane” refers to a basal side layer produced by epithelial cells, is rich in laminin and IV-type collagen, and has a thickness of 50 to 100 nm. The “apical surface” refers to the surface (upper surface layer) formed on the opposite side to the “basal membrane”. In an embodiment, in the retinal tissue developed to the extent that photoreceptor cells or photoreceptor progenitor cells are observed, the “apical surface” refers to a surface in contact with photoreceptor layer (outer nuclear layer) in which outer limiting membrane is formed and photoreceptor cells and photoreceptor progenitor cells are present. Such an apical surface can be identified by, for example, immunostaining (known to those skilled in the art) using an antibody against an apical surface marker (e.g., atypical PKC (hereinafter, abbreviated to “aPKC”), E-cadherin, N-cadherin).

### [Method for Producing Retinal Cell or Retinal Tissue]

The present invention provides, as an aspect of the present invention, a method for producing a retinal cell or retinal tissue. The production method comprises the following steps:
(A) suspension-culturing a pluripotent stem cell to form a cell aggregate of pluripotent stem cells; and
(B) suspension-culturing the cell aggregate obtained in the step (A) in the presence of a BMP signaling pathway agonist and a CHK1 signaling pathway inhibitor to obtain a cell aggregate containing a retinal cell.

The production method of the present invention is a method for producing a retinal cell or retinal tissue by differentiating pluripotent stem cells with a combination of a BMP signaling pathway agonist and a CHK1 signaling pathway inhibitor. As methods for differentiating pluripotent stem cells into retinal cells or retinal tissue, methods disclosed in WO2011/055855, WO2013/077425, WO2015/025967, WO2016/063985, WO2016/063986, WO2017/183732, PLOS One. 2010 Jan. 20; 5 (1): e8763., Stem Cells. 2011 August; 29 (8): 1206-18., Proc Natl Acad Sci USA. 2014 Jun 10; 111 (23): 8518-23, and Nat Commun. 2014 Jun. 10; 5: 4047 are known, but there was no method for producing a retinal cell or retinal tissue with a CHK1 signaling pathway inhibitor.

Pluripotent stem cells are as described above, and examples of preferable pluripotent stem cells include induced pluripotent stem cells or ES cells, further preferably human induced pluripotent stem cells or human ES cells. The method for producing induced pluripotent stem cells is not particularly limited, and induced pluripotent stem cells can be produced by methods known to those skilled in the art, but it is desirable to perform preparing induced pluripotent stem cells (i.e., reprogramming somatic cells to establish pluripotent stem cells) under feeder-free conditions, too.

### <Step (A)>

The step (A) is a step of suspension-culturing a pluripotent stem cell to form a cell aggregate of pluripotent stem cells. The pluripotent stem cell to be used in the step (A) can be obtained by maintenance culture/expansion culture. It follows that the step (A) may include (A-1) maintenance-culturing/expansion-culturing a pluripotent stem cell and (A-2) suspension-culturing a pluripotent stem cell obtained in the step (A-1) to form a cell aggregate of pluripotent stem cells. Maintenance culture/expansion culture to obtain a pluripotent stem cell can be performed by methods known to those skilled in the art. Maintenance culture/expansion culture of pluripotent stem cells can be performed by adherent culture or suspension culture, but is preferably performed by adherent culture. Maintenance culture/expansion culture of pluripotent stem cells may be performed in the presence of a feeder, or under feeder-free conditions, but is preferably performed under feeder-free conditions. Under feeder-free conditions, culture media containing a factor for maintaining undifferentiated state mentioned later can be used.

The culture medium in the step (A-1) may further involve a TGFβ family signaling pathway inhibitor and/or a sonic hedgehog signaling pathway agonist. Also, culture medium in the step (A-2) may involve a sonic hedgehog signaling pathway agonist and/or a Wnt signaling pathway inhibitor, as mentioned later. This method is also disclosed in, for example, WO2015/025967, WO2016/063985, and WO2017/183732. For more details, see WO2015/025967, WO2016/063985, WO2017/183732, etc.

The culture medium that is used in the preparation of the cell aggregate can employ a basal medium for cell proliferation (also referred to as a basal medium), unless otherwise specified. The basal medium for cell proliferation is not particularly limited as long as the culture of cells is possible. A basal medium commercially available as a culture medium for cell proliferation can be appropriately used. Specifically, examples thereof can include culture media that can be used in the culture of animal cells, such as BME medium, BGJb medium, CMRL 1066 medium, Glasgow MEM (GMEM) medium, Improved MEM Zinc Option medium, IMDM medium, Medium 199 medium, MEM medium, Eagle MEM medium, αMEM medium, DMEM medium, F-12 medium, DMEM/F12 medium, IMDM/F12 medium, Ham's medium, RPMI 1640 medium, Fischer's medium, Leibovitz's L-15 medium and mixtures of these media. Alternatively, a culture medium supplemented with N2 medium which is an assisted culture medium may be used.

The TGFβ family signaling pathway inhibitor refers to a substance inhibiting the TGFβ family signaling pathway, i.e., the signaling pathway transduced by the Smad family. Specifically, examples thereof can include TGFβ signaling pathway inhibitors (e.g., SB431542, LY-364947, SB505124, A-83-01), Nodal/activin signaling pathway inhibitors (e.g., SB431542, A-83-01) and BMP signaling pathway inhibitors (e.g., LDN193189, dorsomorphin). These substances are commercially available and can be obtained.

The sonic hedgehog (hereinafter, also referred to as “Shh”) signaling pathway agonist is a substance capable of enhancing signal transduction mediated by Shh. Examples of the Shh signaling pathway agonist include SHH, partial peptides of SHH, PMA (purmorphamine), and SAG (smoothened agonist).

The concentrations of the TGFβ family signaling pathway inhibitor and the sonic hedgehog signaling pathway agonist can be concentrations capable of inducting differentiation into retinal cells. For example, SB431542 is used at a concentration of usually 0.1 to 200 μM, preferably 2 to 50 μM. A-83-01 is used at a concentration of usually 0.05 to 50 μM, preferably 0.5 to 5 μM. LDN193189 is used at a concentration of usually 1 to 2000 nM, preferably 10 to 300 nM. SAG is used at a concentration of usually 1 to 2000 nM, preferably 10 to 700 nM. PMA is used at a concentration of usually 0.002 to 20 μM, preferably 0.02 to 2 μM.

The factor for maintaining undifferentiated state is not particularly limited as long as it is a substance having an action of suppressing the differentiation of pluripotent stem cells. Examples of the factor for maintaining undifferentiated state that is generally used by those skilled in the art can include FGF signaling pathway agonists, TGFβ family signaling pathway agonists, and insulin. Examples of the FGF signaling pathway agonist specifically include fibroblast growth factors (e.g., bFGF, FGF4, FGF8, further preferably bFGF). Examples of the TGFβ family signaling pathway agonist include TGFβ signaling pathway agonists and Nodal/activin signaling pathway agonists. Examples of the TGFβ signaling pathway agonist include TGFβ1 and TGFβ 2. Examples of the Nodal/activin signaling pathway agonist include Nodal, activin A, and activin B. In the case of culturing human pluripotent stem cells (human ES cells, human iPS cells), the culture medium in the step (A-1) preferably contains bFGF as the factor for maintaining undifferentiated state.

The concentration of the factor for maintaining undifferentiated state in the culture medium that is used in the step (A-1) is a concentration capable of maintaining the undifferentiated state of the pluripotent stem cells to be cultured, and can be appropriately set by those skilled in the art. For example, specifically, in the case of using bFGF as the factor for maintaining undifferentiated state in the absence of feeder cells, its concentration is usually on the order of 4 ng to 500 ng/ml, preferably on the order of 10 ng to 200 ng/mL, more preferably on the order of 30 ng to 150 ng/mL.

Many synthetic media have been developed or are commercially available as feeder-free media that contain a factor for maintaining undifferentiated state and can be used for culturing pluripotent stem cells, and examples thereof include Essential 8 medium (manufactured by Life Technologies Corp.). The Essential 8 medium contains L-ascorbic acid-2-phosphate magnesium (64 mg/L), sodium selenium (14 μg/L), insulin (19.4 mg/L), NaHCO₃ (543 mg/L), transferrin (10.7 mg/L), bFGF (100 ng/mL), and the TGFβ family signaling pathway agonist (TGFβ1 (2 ng/ml) or Nodal (100 ng/mL)) as additives in DMEM/F12 medium (Nature Methods, 8, 424-429 (2011)). Examples of other commercially available feeder-free media include S-medium (manufactured by DS Pharma Biomedical Co., Ltd.), StemPro (manufactured by Life Technologies Corp.), hESF9 (Proc. Natl. Acad. Sci. USA. 2008 Sep. 9; 105 (36): 13409-14), mTeSRI (manufactured by STEMCELL Technologies Inc.), mTeSR2 (manufactured by STEMCELL Technologies Inc.), TeSR-E8 (manufactured by STEMCELL Technologies Inc.), and StemFit (manufactured by Ajinomoto Co., Inc.). In the step (A-1), the present invention can be conveniently carried out by using these. By using these culture media, it is possible to perform the culture of pluripotent stem cells under feeder-free conditions. The culture medium that is used in the step (A-1) is, as one example, a serum-free medium that is not supplemented with any of the BMP signaling pathway agonist, the Wnt signaling pathway agonist and the Wnt signaling pathway inhibitor.

In the culture of pluripotent stem cells under feeder-free conditions in the step (A-1), a suitable matrix may be used as a scaffold in order to provide a scaffold as a replacement for feeder cells to the pluripotent stem cells. Examples of the matrix that can be used as a scaffold include laminin (Nat Biotechnol 28, 611-615, (2010)), laminin fragments (Nat Commun 3, 1236, (2012)), basal membrane preparations (Nat Biotechnol 19, 971-974, (2001)), gelatin, collagen, heparan sulfate proteoglycan, entactin, and vitronectin.

The culture time of the pluripotent stem cells in the step (A-1) is not particularly limited within a range in which an effect of improving the quality of the cell aggregate to be formed in the step (A-2) can be achieved in the case of culture in the presence of the TGFβ family signaling pathway inhibitor and/or the sonic hedgehog signaling pathway agonist (e.g., from 100 nM to 700 nM), and is usually from 0.5 to 144 hours. In an embodiment, it is preferably from 2 to 96 hours, more preferably from 6 to 48 hours, further preferably from 12 to 48 hours, still further preferably from 18 to 28 hours (e.g., 24 hours).

In the case of culture in the presence of the TGFβ family signaling pathway inhibitor and/or the sonic hedgehog signaling pathway agonist (e.g., 100 nM to 700 nM), cells to be obtained in the step (A-1) are cells with the pluripotent-like state maintained, and the pluripotent-like state is maintained throughout the step (A-1). The pluripotent-like state means a state in which at least some of the traits that are unique to pluripotent stem cells and common among pluripotent stem cells, including pluripotency, are maintained. Strict pluripotency is not required for the pluripotent-like state. Specifically, a state in which all or some of the markers that serve as indexes of pluripotent state are expressed is included in the “pluripotent-like state”. Examples of the markers for pluripotent-like state include Oct3/4 positiveness and alkaline phosphatase positiveness. In an embodiment, cells with the pluripotent-like state maintained are Oct3/4-positive. Even in the case that the expression level of Nanog is lower than that in ES cells or iPS cells, the case corresponds to “cells that exhibit pluripotent-like state”. In an embodiment, cells to be obtained in the step (A-1) include Oct3/4-positive stem cells in a proportion of 60% or more, for example, 90% or more.

In the step (A-2), the pluripotent stem cell obtained in the step (A-1) is suspension-cultured to form a cell aggregate of pluripotent stem cells. In the “cell aggregate of pluripotent stem cells” here, not all the cells need to be pluripotent stem cells, and the case that a certain proportion (e.g., 50% or more, 60% or more, 70% or more, 80% or more, 90% or more) of pluripotent stem cells are contained is also included. In the case of culture in the presence of the TGFβ family signaling pathway inhibitor and/or the sonic hedgehog signaling pathway agonist (e.g., 100 nM to 700 nM) in the step (A-1), as one example, a cell aggregate of the “cells that exhibit pluripotent-like state” described above (e.g., containing Oct3/4-positive stem cells in a proportion of 60% or more, for example, 90% or more) is formed, and this is also encompassed in the “cell aggregate of pluripotent stem cells”.

Suspension culture is culturing cells in a state non-adhesive to a culture vessel, and can be performed, without particular limitation, by using a culture vessel that has not been artificially treated (e.g., coating treatment with an extracellular matrix or the like) for the purpose of enhancing the adhesion to cells, or a culture vessel subjected to coating treatment by treatment to artificially inhibit adhesion (e.g., water-soluble polymer containing polyhydroxyethylmethacrylic acid (poly-HEMA), nonionic surface-active polyol (e.g., Pluronic F-127) or 2-methacryloyloxyethyl phosphorylcholine as constituent units (Lipidure)).

Suspension culture can be performed by using an SFEB (Serum-free Floating culture of Embryoid Bodies-like aggregates) method (WO2005/12390) or an SFEBq method (WO2009/148170).

The culture medium that is used in the step (A-2) may be a serum-containing medium or a serum-free medium. A serum-free medium is suitably used from the viewpoint of circumventing contamination with chemically undetermined components. In order to circumvent the complication of preparation, examples thereof include serum-free media supplemented with an appropriate amount of a serum replacement such as commercially available KSR. The amount of KSR added to the serum-free medium is usually from about 1% to about 30%, preferably from about 2% to about 20%.

For the formation of the aggregate, first, dispersed cells are prepared by the dispersion operation of the cells obtained in the step (A-1). The “dispersed cells” obtained by dispersion operation include a state in which 70% (preferably 80% or more) or more are single cells and 30% or less (preferably 20% or less) of 2- to 50-cell masses are present. The dispersed cells include a state in which the mutual adhesion (e.g., surface adhesion) of cells has been mostly lost.

A suspension of the dispersed cells is seeded into an incubator, and the dispersed cells are cultured under conditions of non-adhesive to the incubator, thereby causing the aggregation of a plurality of cells to form an aggregate. In an embodiment, when a predetermined number of dispersed stem cells is placed in each well of a multi-well plate (U-bottom, V-bottom) such as a 96-well plate and this is statically cultured, the cells aggregate rapidly, thereby forming one aggregate in each well (SFEBq). In the case of suspension-culturing cells using a 96-well plate, a liquid prepared so as to attain about 1×10³ to about 1×10⁵ cells (preferably about 3×10³ to about 5×10⁴ cells or about 4×10³ to about 2×10⁴ cells) per well is added to the wells, and the plate is left standing to form aggregates.

In an embodiment, the culture medium that is used in the step (A-2) contains a sonic hedgehog signaling pathway agonist. In other words, in a specific embodiment, the step (A) includes the following step (A1) and step (A2):
(A1) culturing pluripotent stem cells in a culture medium containing a factor for maintaining undifferentiated state and optionally containing a TGFβ family signaling pathway inhibitor and/or a sonic hedgehog signaling pathway agonist in the absence of feeder cells; and
(A2) suspension-culturing the cells obtained in the step (A1) in a culture medium containing a sonic hedgehog signaling pathway agonist to form a cell aggregate.

As the sonic hedgehog signaling pathway agonist in the step (A-2), the one mentioned above can be used at the concentration mentioned above (e.g., from 10 nM to 300 nM). The sonic hedgehog signaling pathway agonist is preferably contained in the culture medium from the start of suspension culture. A ROCK inhibitor (e.g., Y-27632) may be added to the culture medium. The culture time is, for example, from 12 hours to 6 days. The culture medium that is used in the step (A-2) is, as one example, a culture medium that is not supplemented with one or more (preferably all) selected from the group consisting of a BMP signaling pathway agonist, a Wnt signaling pathway agonist, a TGFβ family signaling pathway inhibitor and a TGFβ family signaling pathway agonist.

### <Step (B)>

The step (B) is a step of suspension-culturing the cell aggregate obtained in the step (A) in the presence of a BMP signaling pathway agonist and a CHK 1 signaling pathway inhibitor to obtain a cell aggregate containing a retinal cell.

Examples of the culture medium that is used in the step (B) include serum-free media or serum media (preferably serum-free media) supplemented with a BMP signaling pathway agonist and a CHK1 signaling pathway inhibitor. The serum-free medium and the serum medium can be provided as mentioned above. The culture medium that is used in the step (B) is, as one example, a culture medium that is not supplemented with one or more (preferably all) selected from the group consisting of a Wnt signaling pathway agonist, a TGFβ family signaling pathway inhibitor and a TGFβ family signaling pathway agonist. Alternatively, the culture medium that is used in the step (B) is, as one example, a culture medium that is not supplemented with a sonic hedgehog signaling pathway agonist. Alternatively, the culture medium that is used in the step (B) is a culture medium that may be supplemented with a Wnt signaling pathway agonist.

The BMP signaling pathway agonist is a substance capable of enhancing the signaling pathway mediated by BMP. Examples of the BMP signaling pathway agonist include BMP protein such as BMP2, BMP4 and BMP7, GDF protein such as GDF7, anti-BMP receptor antibodies, and BMP partial peptides. The BMP2 protein, the BMP4 protein and the BMP7 protein are available from, for example, R&D Systems, Inc., and the GDF7 protein is available from, for example, Wako Pure Chemical Industries, Ltd. The BMP signaling pathway agonist is preferably one or more proteins selected from the group consisting of BMP2, BMP4, BMP7 and GDF7.

The typical concentration of the BMP signaling pathway agonist in the culture medium can be a concentration capable of inducing differentiation into retinal cells. Examples of the typical concentration of human BMP4 protein capable of inducing differentiation into retinal cells when being used not in combination with a CHK1 signaling pathway inhibitor include concentrations of about 0.1 nM or more (more than 0.15 nM), preferably about 0.5 nM or more, more preferably about 1 nM or more, further preferably about 1.5 nM (55 ng/ml), or, about 1 μM or less, preferably about 100 nM or less, more preferably about 10 nM or less, further preferably about 5 nM or less. The typical concentrations of other BMP signaling pathway agonists can be each a concentration having a BMP signaling pathway-activating action comparable to that of human BMP4 protein at the above concentration. The concentrations can be readily set by those skilled in the art. Specifically, the BMP activity can be determined by measuring the alkaline phosphatase production-inducing ability of ATDC-5 cells, which are mouse cartilaginous progenitor cells.

In an embodiment of the present invention, the concentration of the human BMP4 protein in using in combination with a CHK1 signaling pathway inhibitor is such a concentration that an almost truly spherical cell aggregate is formed and differentiation into a retinal pigment epithelial cell is suppressed. Specifically, the concentration of the human BMP4 protein in the culture medium may be a concentration as low as about 1/10 of the above typical concentration, specifically, may be about 0.001 nM, preferably about 0.01 nM, more preferably about 0.1 nM, further preferably about 0.15 nM (5.5 ng/ml), or, about 0.1 μM or less, preferably about 10 nM or less, more preferably about 1 nM or less (less than 1.5 nM), further preferably about 0.5 nM or less. In this case, a cell aggregate having more favorable shape (being almost truly spherical) can be obtained. In addition, differentiation into non-target cells such as retinal pigment epithelial (RPE) cells is suppressed, and a cell aggregate containing few non-target cells can be obtained. Furthermore, the cell aggregate is very favorable as an intermediate for producing a neural retina including a ciliary marginal zone-like structure (corresponding to steps (C) and (D) of a production method mentioned later).

In an embodiment of the present invention, the concentration of the human BMP4 in using in combination with a CHK1 signaling pathway inhibitor is such a concentration that a sphere-like cell aggregate comprising a multilayer structure of an outer structure including a retinal pigment epithelial cell and an inner structure including a neural retina is formed. Specifically, the concentration of the human BMP4 protein in the culture medium may be, for example, about 0.01 nM or more, preferably about 0.1 nM or more, more preferably about 0.5 nM or more, further preferably about 1 nM or more, most preferably about 1.5 nM (55 ng/mL), or, about 1 μM or less, preferably about 100 nM or less, more preferably about 10 nM or less, further preferably about 5 nM or less. In the case of using other BMP signaling pathway agonists, the concentrations can be each a concentration having a BMP signaling pathway-activating action comparable to that of the human BMP4 protein in the concentration mentioned above.

The CHK1 signaling pathway inhibitor is a substance that inhibits a signaling pathway mediated by CHK1 (checkpoint kinase 1). In an embodiment, the signaling pathway mediated by CHK1 is a signaling pathway via CHK1-p21-CDK9 (cyclin-dependent kinase 9). Specifically, the signaling pathway is a series of signaling pathways in which CHK1 activates p21, and the activated p21 inhibits CDK9 to inhibit the phosphorylation of SMAD2/3, suppressing the decomposition of SMAD2/3. Accordingly, examples of the CHK1 signaling pathway inhibitor include a CHK1 inhibitor, a p21 inhibitor or a CDK9 activator.

An embodiment of the CHK1 signaling pathway inhibitor is, for example, a CHK1 inhibitor that binds to CHK1 to inhibit the activity of CHK1. Specific examples of the CHK1 inhibitor include, but are not limited to, PD407824 (CAS622864-54-4), CHIR-124 (CAS405168-58-3), debromohymenialdisine (CAS75593-17-8), SB 218078 (CAS135897-06-2), the Chk2-suppressing agent (CAS724708-21-8) LY2603618 (CAS911222-45-2), SCH 900776 (CAS891494-63-6), TCS 2312 (CAS838823-32-8), PF 477736 (CAS952021-60-2), UCN-01 (CAS112953-11-4), AZD7762 (CAS860352-01-8), XL844 (CAS: NONE), CBP501 (CAS565434-85-7), and an anti-CHK1 antibody. siRNA for CHK1, which decomposes mRNA for CHK1, is also included in CHK1 inhibitors. The CHK1 signaling pathway inhibitor is preferably PD407824 (CAS622864-54-4) having a structure shown in the following:

### [Chemical Formula 1]

An embodiment of the CHK1 signaling pathway inhibitor is, for example, a p21 inhibitor that binds to p21 to inhibit the activity of p21. Specific examples of the p21 inhibitor include an anti-p21 antibody and siRNA for p21. An embodiment of the CHK1 signaling pathway inhibitor is, for example, a CDK9 activator that binds to CDK9 to enhance the activity of CDK9.

The concentration of the CHK1 signaling pathway inhibitor may be a concentration capable of enhancing the differentiating action of the BMP signaling pathway agonist, and, for PD407824, can be a concentration of about 0.1 μM to about 10 μM, about 0.3 μM to about 10 μM, about 0.5 μM to about 10 μM, about 0.5 μM to about 5 μM, or about 1 μM to about 5 μM. The concentrations of other CHK1 signaling pathway inhibitors may be each a concentration that exerts a CHK1 signaling pathway inhibitory effect (e.g., CHK1 inhibitory effect) comparable to that of PD407824 in a concentration of about 0.1 μM to about 10 μM, about 0.3 μM to about 10 μM, about 0.5 μM to about 10 μM, about 0.5 μM to about 5 μM, or about 1 μM to about 5 μM. The CHK1 inhibitory effect can be appropriately examined, for example, by using an approach known to those skilled in the art or a commercially available reagent or kit (e.g., Promega Corporation, catalog No.: V1941). Specifically, a peptide derived from human CDC25C (KKKVSRSGLYRSPSMPENLNRPR (SEQ ID NO: 1)), CHK1, and an evaluation target substance are mixed together in a reaction container containing ATP, and the degree of phosphorylation of the peptide can be detected. The degree of phosphorylation of SMAD2/3 present in the downstream of the pathway may be detected as the inhibitory effect for the CHK1 signaling pathway by using a commercially available anti-phosphorylated SMAD2/3 antibody or the like.

The BMP signaling pathway agonist can be added about 24 hours or later after the start of suspension culture in the step (A), and may be added to the culture medium within several days (e.g., within 15 days) after the start of suspension culture. Preferably, the BMP signaling pathway agonist is added to the culture medium between Day 1 and Day 15, more preferably between Day 1 and Day 9, or between Day 2 and Day 9, most preferably on Day 3, after the start of suspension culture in the step (A).

After differentiation of cells forming an aggregate into retina cells has been initiated through the addition of the BMP signaling pathway agonist to the culture medium, it is not needed to add the BMP signaling pathway agonist to the culture medium, and the culture medium may be exchanged with a serum-free medium or serum medium containing no BMP signaling pathway agonist. In an embodiment, after the differentiation into retina cells has been initiated, the concentration of the BMP signaling pathway agonist in the culture medium is decreased gradually or in stages at a decrease rate of 40 to 60% per 2 to 4 days by exchanging the culture medium with a serum-free medium or serum medium containing no BMP signaling pathway agonist. The cells for which differentiation into retina cells has been initiated can be confirmed, for example, by detecting expression of a retinal progenitor cell marker gene (e.g., Rx gene (also called Rax), Pax6 gene, Chx10 gene) in the cells. Alternatively, the timing at which differentiation into retina cells was initiated can be confirmed by detecting fluorescence emitted from a fluorescent reporter protein expressed through suspension culture of an aggregate in the presence of the BMP signaling pathway agonist in a concentration required for differentiation into retina cells, wherein the aggregate has been formed in the step (2) with pluripotent stem cells with a gene for fluorescent reporter protein such as GFP knocked in the Rx locus. An embodiment of the step (3) can be, for example, a step of suspension-culturing the aggregate formed in the step (2) in a serum-free medium or serum medium containing the BMP signaling pathway agonist in a concentration required for differentiation into retia cells until cells expressing a retinal progenitor cell marker gene (e.g., Rx gene, Pax6 gene, Chx10 gene) begin to appear to obtain an aggregate containing a retinal progenitor cell.

In a specific embodiment, the BMP signaling pathway agonist is added to the culture medium, for example, between Days 1 and 9, preferably between Days 2 and 9, after the start of suspension culture in the step (B). In the case that the BMP signaling pathway agonist is BMP4, for example, a part or the whole of the culture medium is exchanged with a culture medium containing BMP4 between Days 2 and 9 after the start of suspension culture in the step (B) to adjust the final concentration of BMP4 to about 1 to 10 nM, and culture can be performed for, for example, 1 to 12 days, preferably 2 to 9 days, further preferably 2 to 5 days, in the presence of BMP4. In this context, in order to maintain the concentration of BMP4 at the same concentration, a part or the whole of the culture medium can be exchanged with a culture medium containing BMP4 once or about twice. Alternatively, the concentration of BMP4 may be decreased in stages. For example, the concentration of the BMP signaling pathway agonist (BMP4) is maintained from Days 2 to 10 after the start of suspension culture in the step (B), and then, the concentration of the BMP signaling pathway agonist (BMP4) may be decreased in stages from Days 6 to 20 after the start of suspension culture in the step (B).

The CHK1 signaling pathway inhibitor can coexist with the BMP signaling pathway agonist in the culture medium for a certain period in the step (B), is preferably added to the culture medium simultaneously with the BMP signaling pathway agonist, and preferably coexists with the BMP signaling pathway agonist for the same period as the period during which the BMP signaling pathway agonist is added.

Culture conditions such as culture temperature and CO₂ concentration in the step (A) to the step (B) can be appropriately set. The culture temperature is, for example, from about 30° C. to about 40° C., preferably about 37° C. The CO₂ concentration is, for example, from about 1% to about 10%, preferably about 5%.

Retinal cells at various stages of differentiation can be produced as retinal cells contained in the cell aggregate by varying the culture period in the step (B). In other words, retinal cells in the cell aggregate containing immature retinal cells (e.g., retinal progenitor cell, photoreceptor progenitor cell) and matured retinal cells (e.g., photoreceptor cell) at various ratios can be produced. The ratio of matured retinal cells can be increased by extending the culture period in the step (B).

In the step (A) and/or the step (B), a Wnt signaling pathway inhibitor may be further added to the culture medium.

The Wnt signaling pathway inhibitor that is used in the step (A) and/or the step (B) is not particularly limited as long as it is capable of suppressing signal transduction mediated by Wnt, and may be any of a protein, a nucleic acid, a low-molecular compound, and the like. Signals mediated by Wnt are transduced via Wnt receptor present as a heterodimer of frizzled (Fz) and LRP5/6 (low-density lipoprotein receptor-related protein 5/6). Examples of the Wnt signaling pathway inhibitor include, but are not limited to, substances acting directly on Wnt or Wnt receptor (anti-Wnt neutralizing antibody, anti-Wnt receptor neutralizing antibody, etc.), substances suppressing the expression of a gene encoding Wnt or Wnt receptor (e.g., antisense oligonucleotide, siRNA), substances inhibiting the binding of Wnt to Wnt receptor (soluble Wnt receptor, dominant negative Wnt receptor, etc., Wnt antagonist, Dkk1, Cerberus protein, etc.), and substances inhibiting bioactivity caused by signal transduction ascribable to Wnt receptor [e.g., low-molecular compounds such as CKI-7 (N-(2-aminoethyl)-5-chloroisoquinoline-8-sulfonamide), D4476 (4-[4-(2,3-dihydro-1,4-benzodioxin-6-yl)-5-(2-pyridinyl)-1H-imidazol-2-yl]benzamide), IWR-1-endo (IWR1e) (4-[(3aR,4S,7R,7aS)-1,3,3a,4,7,7a-hexahydro-1,3-dioxo-4,7-methano-2H-isoindol-2-yl]-N-8-quinolinyl-benzamide), and IWP-2 (N-(6-methyl-2-benzothiazolyl)-2-[(3,4,6,7-tetrahydro-4-oxo-3-phenylthieno[3,2-d]pyrimidin-2-yl)thio]acetamide)]. One or two or more of these may be contained as the Wnt signaling pathway inhibitor. CKI-7, D4476, IWR-1-endo (IWR1e), IWP-2, and the like are known Wnt signaling pathway inhibitors, and commercially available products, etc. can be appropriately obtained. IWR1e is preferably used as the Wnt signaling pathway inhibitor.

The concentration of the Wnt signaling pathway inhibitor in the step (A) can be a concentration capable of inducing the favorable formation of the cell aggregate. For example, IWR-1-endo is added to the culture medium so as to attain a concentration of about 0.1 μM to about 100 μM, preferably about 0.3 μM to about 30 μM, more preferably about 1 μM to about 10 μM, further preferably about 3 μM. In the case of using a Wnt signaling pathway inhibitor other than IWR-1-endo, it is desirable to be used at a concentration that exhibits Wnt signaling pathway inhibitory activity equivalent to the concentration of IWR-1-endo.

In the step (A), the timing of adding the Wnt signaling pathway inhibitor to the culture medium is preferably earlier. The Wnt signaling pathway inhibitor is added to the culture medium usually within 6 days, preferably within 3 days, more preferably within 1 day, more preferably within 12 hours, from the start of suspension culture in the step (A), further preferably at the start of suspension culture in the step (A). Specifically, for example, the addition of a basal medium supplemented with the Wnt signaling pathway inhibitor, or the exchange of a part or the whole of the culture medium with the basal medium can be performed. Although a period for which the Wnt signaling pathway inhibitor is allowed to act on the cells obtained through maintenance culture/expansion culture in the step (A) is not particularly limited, it is preferably added to the culture medium at the start of suspension culture in the step (A) and then allowed to act until the completion of the step (A) (immediately before addition of a BMP signaling pathway agonist). Further preferably, as mentioned later, exposure to the Wnt signaling pathway inhibitor is continued even after the completion of the step (A) (i.e., during the period of the step (B)). In an embodiment, as mentioned later, the action of the Wnt signaling pathway inhibitor is continued even after the completion of the step (A) (i.e., during the period of the step (B)), and the action may be performed until retinal tissue is formed.

In the step (B), as the Wnt signaling pathway inhibitor, any of the Wnt signaling pathway inhibitors mentioned above can be used, whereas, preferably, the same type as the Wnt signaling pathway inhibitor used in the step (A) is used in the step (B).

The concentration of the Wnt signaling pathway inhibitor in the step (B) can be a concentration capable of inducing retinal progenitor cells and retinal tissue. For example, IWR-1-endo is added to the culture medium so as to attain a concentration of about 0.1 μM to about 100 μM, preferably about 0.3 μM to about 30 μM, more preferably about 1 μM to about 10 μM, further preferably about 3 μM. In the case of using a Wnt signaling pathway inhibitor other than IWR-1-endo, it is desirable to be used at a concentration that exhibits Wnt signaling pathway inhibitory activity equivalent to the concentration of IWR-1-endo. The concentration of the Wnt signaling pathway inhibitor in the culture medium in the step (B) is preferably 50 to 150, more preferably 80 to 120, further preferably 90 to 110, when the concentration of the Wnt signaling pathway inhibitor in the culture medium in the step (A) is defined as 100, and it is more preferable to be equivalent to the concentration of the Wnt signaling pathway inhibitor in the culture medium in the step (B).

The timing of addition of the Wnt signaling pathway inhibitor to the culture medium is not particularly limited within a range that can achieve the formation of a cell aggregate containing retinal cells or retinal tissue, and is preferably earlier. Preferably, the Wnt signaling pathway inhibitor is added to the culture medium at the start of the step (B). More preferably, the Wnt signaling pathway inhibitor is added in the step (A) and then also continuously (i.e., from the start of the step (A)) contained in the culture medium in the step (B). Further preferably, the Wnt signaling pathway inhibitor is added at the start of suspension culture in the step (A) and then also continuously contained in the culture medium in the step (B). For example, a BMP signaling pathway agonist (e.g., BMP4) can be added to the cultures (suspension of aggregates in a culture medium containing a Wnt signaling pathway inhibitor) obtained in the step (A).

A period for which the Wnt signaling pathway inhibitor is allowed to act is not particularly limited, but is preferably from 2 days to 30 days, more preferably from 6 days to 20 days, from 8 days to 18 days, from 10 days to 18 days, or from 10 days to 17 days (e.g., 10 days), with the start of suspension culture in the step (A) as a commencement when the Wnt signaling pathway inhibitor is added at the start of suspension culture in the step (A). In another embodiment, the period for which the Wnt signaling pathway inhibitor is allowed to act is preferably from 3 days to 15 days (e.g., 5 days, 6 days, 7 days), more preferably from 6 days to 10 days (e.g., 6 days), with the start of suspension culture in the step (A) as a commencement when the Wnt signaling pathway inhibitor is added at the start of suspension culture in the step (A).

A neural retina having a ciliary marginal zone-like structure can also be produced by culturing the cell aggregate obtained by the method mentioned above in a serum-free medium or a serum medium containing a Wnt signaling pathway agonist and/or a FGF signaling pathway inhibitor for a period on the order of 2 days to 4 days (step (C)), followed by culture in a serum-free medium or a serum medium containing neither a Wnt signaling pathway agonist nor a FGF signaling pathway inhibitor for about 30 days to about 200 days (from 30 days to 150 days, from 50 days to 120 days, from 60 days to 90 days) (step (D)).

In an embodiment, a neural retina having a ciliary marginal zone-like structure can be produced by the step (C) and the step (D) from the cell aggregate obtained in the step (A) and the step (B), the cell aggregate being of Days 6 to 30 or Days 10 to 20 (Day 10, Day 11, Day 12, Day 13, Day 14, Day 15, Day 16, Day 17, Day 18, Day 19 or Day 20) after the start of suspension culture in the step (A).

The Wnt signaling pathway agonist is not particularly limited as long as it is capable of enhancing signal transduction mediated by Wnt. Examples of a specific Wnt signaling pathway agonist can include GSK3β inhibitors (e.g., 6-bromoindirubin-3′-oxime (BIO), CHIR99021, kenpaullone). For example, in the case of CHIR99021, the range of about 0.1 μM to about 100 μM, preferably about 1 μM to about 30 μM, can be included.

The FGF signaling pathway inhibitor is not particularly limited as long as it can inhibit signal transduction mediated by FGF. Examples of the FGF signaling pathway inhibitor include SU-5402, AZD4547, and BGJ398. For example, SU-5402 is added at a concentration of about 0.1 μM to about 100 μM, preferably about 1 μM to about 30 μM, more preferably about 5 μM.

The culture medium that is used in the step (C) is, as one example, a culture medium that is not supplemented with one or more (preferably all) selected from the group consisting of a BMP signaling pathway agonist, a Wnt signaling pathway inhibitor, a SHH signaling pathway agonist, a TGFβ family signaling pathway inhibitor and a TGFβ family signaling pathway agonist.

A part of the step (D) or the whole step can perform culture using a culture medium for continuous epithelial tissue maintenance disclosed in WO2019/017492. Specifically, the continuous epithelium structure of the neural retina can be maintained by culture using a culture medium for continuous epithelial tissue maintenance. One example of the culture medium for continuous epithelial tissue maintenance can include a medium in which Neurobasal medium (e.g., manufactured by Thermo Fisher Scientific Inc., 21103049) is blended with B27 supplement (e.g., Thermo Fisher Scientific Inc., 12587010).

For the culture in the step (D), exchange with the culture medium for continuous epithelial tissue maintenance in stages is preferable for achieving both the differentiation and/or maturation of retinal cells (particularly, photoreceptor cell) and the maintenance of the continuous epithelium structure. For example, culture can be performed using a basal medium for cell proliferation (e.g., a culture medium in which DMEM/F12 medium is supplemented with 10% fetal bovine serum, 1% N2 supplement, and 100 μM taurine) for first 10 days to 30 days, a mixture of a basal medium for cell proliferation and a culture medium for continuous epithelial tissue maintenance (culture medium in which a medium in which DMEM/F12 medium is supplemented with 10% fetal bovine serum, 1% N2 supplement, and 100 μM taurine, and a medium in which Neurobasal medium is supplemented with 10% fetal bovine serum, 2% B27 supplement, 2 mM glutamine, and 100 μM taurine, are mixed at a ratio of 1:3) for next 10 days to 40 days, and a culture medium for continuous epithelial tissue maintenance (e.g., a culture medium in which Neurobasal medium is supplemented with 10% fetal bovine serum, 2% B27 supplement, 2 mM glutamine, and 100 μM taurine) for next 20 days to 140 days.

In a part of the step (D) or the whole step, in the case of using any medium of the basal medium for cell proliferation, the culture medium for continuous epithelial tissue maintenance or a mixture of these media, a thyroid hormone signaling pathway agonist may be further contained. By culture in a culture medium containing a thyroid hormone signaling pathway agonist, the production of a retinal cell aggregate becomes possible in which the ratio of bipolar cells, amacrine cells, ganglion cells or horizontal cells, etc. contained in the neural retina is low and the ratio of photoreceptor progenitor cells has been increased.

In the specification, the thyroid hormone signaling pathway agonist is a substance capable of enhancing signal transduction mediated by thyroid hormone, and is not particularly limited as long as it is capable of enhancing the thyroid hormone signaling pathway. Examples of the thyroid hormone signaling pathway agonist include triiodothyronine (hereinafter, also abbreviated to T3), thyroxin (hereinafter, also abbreviated to T4), and thyroid hormone receptor (preferably TRβ receptor) agonists.

Examples of the thyroid hormone receptor agonist known to those skilled in the art can include compounds such as diphenylmethane derivatives, diaryl ether derivatives, pyridazine derivatives, pyridine derivatives and indole derivatives described in International Publication No. WO 97/21993, International Publication No. WO 2004/066929, International Publication No. WO 2004/093799, International Publication No. WO 2000/039077, International Publication No. WO 2001/098256, International Publication No. WO 2003/018515, International Publication No. WO 2003/084915, International Publication No. WO 2002/094319, International Publication No. WO 2003/064369, Japanese Unexamined Patent Publication No. 2002-053564, Japanese Unexamined Patent Publication No. 2002-370978, Japanese Unexamined Patent Publication No. 2000-256190, International Publication No. WO 2007/132475, International Publication No. WO 2007/009913, International Publication No. WO 2003/094845, International Publication No. WO 2002/051805 or International Publication No. WO 2010/122980.

In the case of using T3 as the thyroid hormone signaling pathway agonist, it can be added to the culture medium so as to attain, for example, the range of 0.1 to 1000 nM. Preferably, examples thereof include concentrations having thyroid hormone signaling enhancing activity that corresponds to T3 with a concentration of 1 to 500 nM; more preferably 10 to 100 nM; further preferably 30 to 90 nM; still more preferably around 60 nM. In the case of using T4 as the thyroid hormone signaling pathway agonist, it can be added to the culture medium so as to attain, for example, the range of 1 nM to 500 μM. Preferably, it is the range of 50 nM to 50 μM; more preferably 500 nM to 5 μM. In the case of using other thyroid hormone receptor agonists, the concentration can exhibit activity equivalent to the agonist activity exhibited by T3 or T4 with the concentration mentioned above.

The culture medium that is used in the step (D) may appropriately contain L-glutamine, taurine, serum, or the like. The culture medium that is used in the step (D) is, as one example, a culture medium that is not supplemented with one or more (preferably all) selected from the group consisting of a BMP signaling pathway agonist, a FGF signaling pathway inhibitor, a Wnt signaling pathway agonist, a Wnt signaling pathway inhibitor, a SHH signaling pathway agonist, a TGFβ family signaling pathway inhibitor and a TGFβ family signaling pathway agonist.

In a specific embodiment of the production method of the present invention, the cell aggregate containing a retinal cell or retinal tissue can be prepared by a method comprising the following steps (A) to (E):
(A) culturing pluripotent stem cells in a culture medium containing a factor for maintaining undifferentiated state and optionally containing a TGFβ family signaling pathway inhibitor and/or a sonic hedgehog signaling pathway agonist in the absence of feeder cells;
(B) forming a cell aggregate by suspension-culturing the cells obtained in the step (A) in a culture medium optionally containing a Wnt signaling pathway inhibitor and/or a sonic hedgehog signaling pathway agonist;
(C) further suspension-culturing the cell aggregate obtained in the step (B) in a culture medium containing a BMP signaling pathway agonist and a CHK1 signaling pathway inhibitor (e.g., CHK1 inhibitor) to obtain a cell aggregate containing a retinal cell or retinal tissue;
(D) culturing the cell aggregate obtained in the step (C) in a serum-free medium or a serum medium containing a Wnt signaling pathway agonist and/or a FGF signaling pathway inhibitor for a period on the order of 2 days to 4 days; and
(E) culturing the cell aggregate obtained in the step (D) in a serum-free medium or a serum medium containing neither a Wnt signaling pathway agonist nor a FGF signaling pathway inhibitor and optionally containing a thyroid hormone signaling pathway agonist for about 30 days to about 200 days.

In a specific embodiment, the cell aggregate containing a retinal cell or retinal tissue can be prepared by a method comprising the following steps (A) to (E):
(A) culturing pluripotent stem cells in a culture medium containing a factor for maintaining undifferentiated state and containing a TGFβ family signaling pathway inhibitor and/or a sonic hedgehog signaling pathway agonist in the absence of feeder cells for 12 hours to 48 hours;
(B) forming a cell aggregate by suspension-culturing the cells obtained in the step (A) in a culture medium containing a Wnt signaling pathway inhibitor and/or a sonic hedgehog signaling pathway agonist for 12 hours to 72 days (24 hours to 48 hours);
(C) further suspension-culturing the cell aggregate obtained in the step (B) in a culture medium containing a BMP signaling pathway agonist and a CHK1 signaling pathway inhibitor (e.g., CHK1 inhibitor) for 8 days to 15 days (10 days to 13 days) to obtain a cell aggregate containing a retinal cell or retinal tissue;
(D) culturing the cell aggregate obtained in the step (C) in a serum-free medium or a serum medium containing a Wnt signaling pathway agonist and/or a FGF signaling pathway inhibitor for 2 days to 4 days; and
(E) culturing the cell aggregate obtained in the step (D) in a serum-free medium or a serum medium containing neither a Wnt signaling pathway agonist nor a FGF signaling pathway inhibitor and optionally containing a thyroid hormone signaling pathway agonist for about 10 days to about 200 days.

In this context, the step (D) may comprise the step of performing culture in a basal medium for cell proliferation for 10 days to 30 days, subsequently performing culture in a mixture of a basal medium for cell proliferation and a culture medium for continuous epithelial tissue maintenance containing a thyroid hormone signaling agonist for 10 days to 40 days, and further performing culture in a culture medium for continuous epithelial tissue maintenance containing a thyroid hormone signaling agonist for 20 days to 140 days.

In an embodiment, the step (D) comprises performing culture in the presence of a thyroid hormone signaling pathway agonist for 20 days to 60 days (30 days to 50 days).

In an embodiment, the culture period from the step (A) to the step (D) is from 70 days to 100 days (from 80 days to 90 days).

In the case of producing retinal tissue by the step (A) to the step (D) mentioned above or by only some of the steps, favorable retinal tissue can be produced by using any of the signaling agonists explicitly mentioned for the steps. Under conditions that hinder the purpose of a step (e.g., in the case that other tissue than retinal tissue is induced), it is recommended that no other signaling agonist be contained. In an embodiment, the culture medium in culturing in each step can contain none of sonic hedgehog signaling pathway activators/inhibitors, TGFβ family signaling pathway activators/inhibitors, BMP signaling pathway activators/inhibitors, CHK1 signaling pathway inhibitors, Wnt signaling pathway activators/inhibitors, FGF signaling pathway activators/inhibitors, Nodal signaling pathway activators/inhibitors, MEK (ERK) activators/inhibitors, PI3k/Akt signaling pathway activators/inhibitors, platelet-derived growth factor (PDGF) signaling pathway activators/inhibitors, vascular endothelial growth factor (VEGF) signaling pathway activators/inhibitors, epidermal growth factor (EGF) signaling pathway activators/inhibitors, Notch signaling pathway activators/inhibitors, integrin signaling pathway activators/inhibitors, and retinoic acid, except the signaling agonists explicitly mentioned for the steps. Needless to say, other signaling agonists may be contained unless the purpose of a step is hindered (e.g., in the case that other tissue than retinal tissue is induced).

In an embodiment, it is preferable that the cell aggregate obtained by the production method of the present invention be a sphere-like cell aggregate, and it is more preferable that the cell aggregate obtained by the production method of the present invention be a cell aggregate having an almost truly spherical shape. The cell aggregate contains a retinal cell, and a cell aggregate in an embodiment is a sphere-like cell aggregate comprising a multilayer structure of an outer structure including a retinal pigment epithelial cell and an inner structure including a neural retina.

The sphere-like cell aggregate means a cell aggregate having a stereoscopic shape close to a spherical shape. The stereoscopic shape close to a spherical shape is a shape having a three-dimensional structure, and examples thereof include a spherical shape that exhibits a circle or an ellipse when projected onto a two-dimensional surface, and a shape formed by overlapping a plurality of spherical shapes (e.g., which exhibits a shape formed by 2 to 4 circles or ellipses overlapping when two-dimensionally projected). The sphere-like cell aggregate may have an almost truly spherical shape.

Herein, the “true sphere” means a complete sphere with the distance from the center to the circumference being constant. The “almost truly spherical shape” means a sphere being almost truly spherical. For example, the difference between the longest distance and shortest distance from the center to the circumference may be within 10%, 5%, or 3% of the longest distance. Simply, determination may be performed by observation with a microscope or the like for a two-dimensionally projected circle. For example, the case that the difference between the longest distance and shortest distance from the center of a two-dimensionally projected circle to the circumference thereof is within 10%, 5%, or 3% of the longest distance is also included. As described above, whether the cell aggregate is a “true sphere” or has an “almost truly spherical shape” is determined. In the case of using the production method herein, a cell aggregate being a “true sphere” or having an “almost truly spherical shape” without overlapping of a plurality of shapes can be obtained with high probability.

In an embodiment of the present invention, a cell aggregate having an almost truly spherical shape that contains retinal tissue and not contains non-target cells (e.g., RPE cells) is provided.

The present invention provides, as an aspect of the present invention, a culture of a cell aggregate containing retinal tissue is provided. Specifically, a culture of a cell aggregate containing retinal tissue, the culture containing:
(1) a cell aggregate having an almost truly spherical shape that contains retinal tissue and not contains non-target cells (e.g., RPE cells); and
(2) a vehicle required for maintaining the survivability of the cell aggregate is provided.

In particular, in the case of using the SFEBq method (WO2009/148170) in forming the cell aggregate of the invention of the present application, typically, culture is performed with a multi-well plate (U-bottom, V-bottom) such as a 96-well plate, and each well of the plate contains one cell aggregate. A well containing no cell aggregate may be present. For example, outermost wells of the plate may contain only a culture medium without a cell aggregate for the purpose of preventing the effect of the evaporation of the culture medium.

Thus, the present invention provides, as an embodiment of the present invention, a culture of a cell aggregate containing retinal tissue, the culture containing:
(1) a multi-well plate (e.g., 192-well, 96-well, 48-well, 24-well, 12-well);
(2) in wells of the plate (at least 50%, 60%, 70%, 80%, 90% or 95% or more of all wells),
   (A) a cell aggregate having an almost truly spherical shape that contains retinal tissue and not contains non-target cells (e.g., RPE cells); and
   (B) a vehicle required for maintaining the survivability of the cell population for transplantation.

The proportion of the number of wells containing a cell aggregate having an almost truly spherical shape that contains retinal tissue to the number of wells containing a cell aggregate may be, for example, 50% or more, 60% or more, 70% or more, 80% or more, 90% or more, 95% or more, 97% or more or 98% or more.

“Not containing non-target cells (e.g., RPE cells)” here means containing substantially no non-target cell (e.g., RPE cells). Specifically, the proportion of non-target cells (e.g., RPE cells) to the total number of cells of a cell aggregate is about 5% or less, about 4% or less, about 3% or less, about 2% or less, about 1% or less, about 0.5% or less, about 0.1% or less. The “non-target cells” means cells other than those contained in a neural retina. Cells of brain and spinal cord tissue and eyeball-related tissue are included in the non-target cells as an example, examples of the cells of brain and spinal cord tissue include cells of the telencephalon (cerebrum), the diencephalon (including the hypothalamus), the midbrain, and the spinal cord, and examples of the eyeball-related tissue include retinal pigment epithelium (RPE), ciliary body, lens and optic stalk (eye stalk and optic nerve tissue).

The “culture” in the present invention means a liquid that contains a vehicle required for maintaining survivability and a cell aggregate, and may contain a biological substance further added or produced from the cell aggregate. Examples of the biological substance include, but are not limited to, cytokines and chemokines.

Examples of the “vehicle required for maintaining survivability” include culture media and physiological buffer solutions, but the “vehicle required for maintaining survivability” is not particularly limited as long as cell aggregates containing retinal tissue survive, and those skilled in the art can select appropriate one. An example thereof is a culture medium prepared by using a culture medium commonly used for the culture of animal cells as a basal medium. Examples of the basal medium can include culture media that can be used in the culture of animal cells, such as BME medium, BGJb medium, CMRL 1066 medium, Glasgow MEM (GMEM) medium, Improved MEM Zinc Option medium, IMDM medium, Medium 199 medium, Eagle MEM medium, αMEM medium, DMEM medium, F-12 medium, DMEM/F12 medium, IMDM/F12 medium, Ham's medium, RPMI 1640 medium, Fischer's medium and mixtures of these media.

In an embodiment, the production method may further comprise dissecting retinal tissue of required size for transplantation from the cell aggregate obtained. For example, dissecting can be performed by using tweezers, a knife, scissors, or the like.

### [Sphere-Like Cell Aggregate]

The sphere-like cell aggregate, as an aspect of the present invention, comprises a multilayer structure of an outer structure including a retinal pigment epithelial cell and an inner structure including a neural retina. The sphere-like cell aggregate is characterized in that
(1) a neural retinal layer including at least a photoreceptor layer is formed in the neural retina in the inner structure, and the photoreceptor layer includes one or more cells selected from the group consisting of a photoreceptor cell, a photoreceptor progenitor cell and a retinal progenitor cell,
(2) the neural retina is present in a folded state in the inner structure,
(3) the retinal pigment epithelial cell in the outer structure is an RPE65-positive cell, an MITF-positive cell, or an RPE65-positive and MITF-positive cell, and
(4) the cell aggregate is free of a lens, a vitreous body, a cornea and a blood vessel.

In an embodiment, the outer structure including a retinal pigment epithelial cell in the sphere-like cell aggregate is the outermost layer structure of the sphere-like cell aggregate, and is continuously or intermittently covering at least a part of the surface of the inner structure including a neural retina. The outer structure is covering preferably 30% or more, more preferably 50% or more, of the surface area of the inner structure. Continuously covering the surface of the inner structure means that the outer structure is present as one continuous mass on the surface of the inner structure, and intermittently covering the surface of the inner structure means that the outer structure is present as two or more continuous masses or layers on the surface of the inner structure and the continuous masses are not connecting to each other. In the case that the outer structure is intermittently covering the surface of the inner structure, it is preferable that the continuous masses be continuously covering 10% or more or 20% or more of the surface area of the core part.
(1) A neural retinal layer including at least a photoreceptor layer is formed in the neural retina in the inner structure, and the photoreceptor layer includes one or more cells selected from the group consisting of a photoreceptor cell, a photoreceptor progenitor cell and a retinal progenitor cell (hereinafter, also referred to as the photoreceptor cells, etc.).

Photoreceptor cells include rod photoreceptor cells and cone photoreceptor cells, and the photoreceptor cells, etc. account for 70% or more, preferably 80% or more, more preferably 90% or more, of all cells present in the photoreceptor layer on the basis of the number of nuclei.

Thirty percent or more, preferably 50% or more, more preferably 80% or more, of the inner structure constitutes a neural retina.
(2) The neural retina is present in a folded state in the inner structure. The positions and number of the folding of the neural retina are not particularly limited, and the inner structure may be folded at only some parts of the inner structure, and may be folded only once or a plurality of times (e.g., twice, three times, four times, five times). A part of the inner structure may include a retinal pigment epithelial cell, a ciliary marginal zone-like structure and a void. The structure of the cell aggregate of the present invention, in which the neural retina is folded in the inner structure, is different from that of the retina in vivo. The neural retina may be forming one continuous epithelium structure, or forming a plurality of epithelium structures. The photoreceptor layer in the neural retina of the inner structure is formed in the outermost (the surface, a face in contact with the outer structure) in at least a part of the inner structure of the cell aggregate. Since the neural retina is present in a folded state in the inner structure, the photoreceptor layer may be formed also in the inside. The photoreceptor cells, etc. are present continuously, i.e., by mutual adhesion, in the tangent direction of the surface of the continuous epithelium structure of the inner structure, and the photoreceptor cells, etc. are present continuously in the tangent direction of the surface of the continuous epithelium structure of the inner structure, thereby forming a photoreceptor layer containing the photoreceptor cells, etc. The tangent direction refers to a direction tangent to the surface of epithelial tissues present in the inner structure of the sphere-like cell aggregate, i.e., a direction along which the photoreceptor cells, etc. in the photoreceptor layer are arranged, and is the direction in parallel to the neural retina or the lateral direction.
(3) The outer structure includes retinal pigment epithelial (RPE) cells in contact with each other, wherein RPE cells also include retinal pigment epithelial progenitor cells, and it is preferable that the RPE cells be RPE65-positive cells, MITF-positive cells, or RPE65-positive and MITF-positive cells. The state in which RPE cells are “in contact with each other” means a state in which one RPE cell is in contact with another RPE cell in the outer structure, and independent single RPE cells being not in contact with another RPE cell do not constitute the outer structure.

In at least a part of the cell aggregate, the basal surface of the retinal pigment epithelial cells may be facing the inner structure, and the basal surface of the neural retina may be facing the outer structure. It follows that the basal surface of the retinal pigment epithelial cells and the basal surface of the neural retina are facing to each other. The structure is a feature different from that of the retina in vivo because the basal surface of retinal pigment epithelial cells and the apical surface of the neural retina are facing to each other in the retina in vivo.
(4) In contrast to retinas in vivo including fetal retinas, which include a lens, a vitreous body, a cornea and a blood vessel, the cell aggregate of the present invention is free of a lens, a vitreous body, a cornea and a blood vessel.

The “lens” is a tissue that plays the role of an optical lens to refract light entering from the outside into an eyeball and focus the light on a retina. Examples of partial structures of the lens include the lens epithelium, the lens nucleus, and the lens capsule. Examples of precursor tissues of the lens include the lens placode and the lens vesicle. The lens placode is a lens precursor tissue consisting of a thickened surface ectodermal cell layer. In embryogenesis, the lens placode is formed by the contact of an optic vesicle with surface ectoderms to cause the thickening of the contact areas. The lens vesicle is a vesicle formed by the invagination of the lens placode. The presence of a lens, a partial structure thereof, or precursor tissue thereof can be confirmed by expression of a marker. Examples of the marker for a lens, a partial structure thereof, or precursor tissue thereof include, but are not limited to, L-Maf (lens precursor tissue) and α, β and γ crystallins (lens).

The “vitreous body” is a transparent gelatinous tissue that is present in the posterior of a lens and filling the lumen, having an action to keep the shape of an eyeball and at the same time disperse external force. The vitreous body is made of moisture and protein (collagen). The presence of a vitreous body can be confirmed by the gelatinous shape.

The “cornea” is a transparent watch glass-like tissue occupying about ⅙ of the anterior of the outer layer of an eyewall. Examples of partial structures of the cornea include the corneal epithelium, Bowman's membrane, the corneal stroma, Descemet's membrane, and the corneal endothelium. The cornea is normally composed of five layers consisting of the corneal epithelium, Bowman's membrane, the corneal stroma, Descemet's membrane, and the corneal endothelium in order from the body surface side. The presence of a cornea, a partial structure thereof, or precursor tissue thereof can be confirmed by expression of a marker. Examples of the marker for a cornea, a partial structure thereof, or precursor tissue thereof include pan-cytokeratin (corneal epithelial precursor tissue), E-cadherin (corneal epithelial precursor tissue), cytokeratin 3 (corneal epithelium), cytokeratin 12 (corneal epithelium), cytokeratin 14 (corneal epithelium), p63 (corneal epithelium), ZO-1 (corneal epithelium), PDGFR-α (corneal stroma, corneal endothelium, or precursor tissue thereof), Pitx2 (corneal stroma and precursor tissue of corneal endothelium), and ABCG2 (corneal stroma and precursor tissue of corneal endothelium).

When a lens, etc. are removed from a fetal retina, an empty space is left in the part, and the tissues are divided by the void. The number of cells (e.g., horizontal cells, amacrine cells, bipolar cells) present in an inner layer (a part with the outermost photoreceptor layer excluded) constituting the neural retinal layer of the core part of the sphere-like cell aggregate, and ganglion cells is smaller than that in retinas in vivo. Specifically, the number is, for example, 80% or less, 70% or less, 60% or less, 50% or less, of the number of cells present in an inner layer of a human fetal retina. When a part of a fetal retina is dissected and cultured, a two-dimensional sheet structure having a thickness is formed, but a stereoscopic sphere-like cell aggregate cannot be formed.

The sphere-like cell aggregate may include a ciliary marginal zone-like structure. The ciliary marginal zone-like structure may be included in the outer structure and/or the inner structure. The ciliary marginal zone-like structure may be included between the retinal pigment epithelial cell and the neural retina with the retinal pigment epithelial cell and the neural retina connecting to each other as an epithelium structure. “Connecting to each other as an epithelium structure” means, for example, a state in which apical-basal polarities are continuously connecting. For example, two or more, three or more, four or more, or five or more ciliary marginal zone-like structures may be included in the sphere-like cell aggregate. A ciliary marginal zone-like structure refers to a structure similar to that of a ciliary marginal zone. Examples of the “ciliary marginal zone (CMZ)” can include a tissue that is present in a boundary region between the neural retina and the retinal pigment epithelium in a retina in vivo, and is a region containing tissue stem cells of the retina (retinal stem cells). The ciliary marginal zone is also called ciliary margin or retinal margin, and the ciliary marginal zone, the ciliary margin and the retinal margin are equivalent tissues. The ciliary marginal zone is known to play an important role in the supply of retinal progenitor cells or differentiated cells to retinal tissue, the maintenance of retinal tissue structure, etc. Examples of a marker gene of the ciliary marginal zone can include Rdh10 gene (positive), Otx1 gene (positive) and Zic1 (positive). It follows that the ciliary marginal zone-like structure may include Rdh10-positive cells, Otx1-positive cells, and/or Zic1-positive cells.

The diameter (maximum diameter) of the sphere-like cell aggregate may be, for example, 0.1 mm or more, 0.2 mm or more, 0.5 mm or more, 1 mm or more, or 2 mm or more, and is, for example, 0.2 mm to 2 mm, 0.5 mm to 2 mm, or 0.5 to 1 mm. Here, the diameter of the sphere-like cell aggregate is measured as the longest of distances from the center of the cell aggregate to the surface thereof.

The sphere-like cell aggregate of the present invention can also be dissected into a proper size with tweezers, a knife, scissors, or the like and then transplanted. The shape after dissection is arbitrary, and examples thereof include a sheet containing a neural retina and retinal pigment epithelial cells (also referred to as a cell sheet or an NR-RPE cell sheet). For example, one cell sheet (e.g., 300 μm in diameter and 50 μm in height) is dissected from one aggregated mass of cells, and one or more such sheets are transplanted according to the area of a region with degeneration of photoreceptor cells or retinal pigment epithelial cells. Those skilled in the art can select the number of cell sheets according to the region dying of degeneration. Even a region in which the basal surface of the layer of retinal pigment epithelial cells and the apical surface of the neural retina are facing to each other, a region including no ciliary marginal zone-like structure, and/or a region in which the neural retina is not folded can be selectively dissected.

The present invention also provides, as an aspect of the present invention, a method for producing a sphere-like cell aggregate, comprising:
(A) suspension-culturing a pluripotent stem cell to form a cell aggregate; and
(B) suspension-culturing the cell aggregate obtained in the step (A) in the presence of a BMP signaling pathway agonist and a CHK1 signaling pathway inhibitor to obtain a sphere-like cell aggregate comprising a multilayer structure of an outer structure including a retinal pigment epithelial cell and an inner structure including a neural retina.
   Here, the step (A) and the step (B), etc. are as described for the method mentioned above for producing a retinal cell or retinal tissue.

### [Pharmaceutical Composition, Treatment Method and Therapeutic Product]

An aspect of the present invention includes a pharmaceutical composition containing the cell aggregate of the present invention (e.g., paragraph [0108], [0111] or [0120]) or a part thereof (a composition for transplantation, transplant tissue or a transplant). The pharmaceutical composition preferably further contains a pharmaceutically acceptable carrier, in addition to the cell aggregate of the present invention or a part thereof. A part of the cell aggregate is a part of the cell aggregate applicable to pharmaceutical compositions, and can be obtained by dissecting retinal tissue of required size for transplantation from the cell aggregate.

The pharmaceutical composition can be used in the treatment of a disease caused by the damage of a neural retina-related cell or a neural retina or the injury of a neural retina. Examples of the disease caused by the damage of a neural retina-related cell or a neural retina include ophthalmic diseases such as retinal degenerative diseases, macular degeneration, age-related macular degeneration, retinitis pigmentosa, glaucoma, corneal diseases, retinal detachment, central serous chorioretinopathy, cone dystrophy, and cone rod dystrophy. Examples of the injury state of a neural retina include a state in which photoreceptor cells die of degeneration.

As the pharmaceutically acceptable carrier, a physiological aqueous solvent (physiological saline, buffer, serum-free medium, etc.) can be used. If necessary, the pharmaceutical composition may be blended with a preservative, a stabilizer, a reducing agent, a tonicity agent, and the like which are usually used in a medicine containing tissues or cells to be transplanted in medical transplantation.

The present invention provides, as an aspect of the present invention, a therapeutic product containing the cell aggregate of the present invention or a part thereof for a disease caused by the damage of a neural retina. One aspect of the present invention includes a method for treating a disease caused by the damage of a neural retina-related cell or a neural retina or the injury of a neural retina, comprising transplanting the cell aggregate of the present invention or a part thereof to a subject in need of transplantation (e.g., subretinally to an eye having the ophthalmic disease). As the therapeutic product for a disease caused by the damage of a neural retina, or in order to make up for a corresponding injured site in the injury state of the neural retina, the cell aggregate of the present invention or a part thereof can be used. The disease caused by the damage of a neural retina-related cell or a neural retina or the injury state of a neural retina can be treated by transplanting the cell aggregate of the present invention or a part thereof to a patient having the disease caused by the damage of a neural retina-related cell or a neural retina, or a patient with the injury state of a neural retina, in need of transplantation, and making up for the damaged neural retina. Examples of a transplantation method include a method of subretinally transplanting sheet-like retinal tissue to an injured site through an incision to an eyeball. Examples of a method for transplantation include a method of performing infusion using a thin tube, and a method of performing transplantation by sandwiching between tweezers, and examples of the thin tube include injection needles.

### EXAMPLES

Hereinafter, the present invention will be described in detail with reference to Examples, but the present invention is by no means limited thereto.

### Example 1 Examination of Differentiation Into Retina by Increasing Sensitivity to BMP4

Human ES cells genetically engineered so as to have Rx::Venus reporter gene (derived from KhES-1 strain (Non Patent Literature 3)) were cultured in the absence of a feeder cell (under feeder-free conditions) in accordance with the method described in “Scientific Reports, 4, 3594 (2014)”. As a culture medium for use in culturing in the absence of a feeder cell (feeder-free medium), StemFit medium (trade name: AK03N, manufactured by Ajinomoto Co., Inc.) was used, and as a scaffold as an alternative to feeder cells, Laminin511-E8 (trade name, manufactured by Nippi, Inc.) was used.

Specific operation of maintenance culture for human ES cells was performed as follows: First, human ES cells that reached sub-confluency (state where about 60% of the culture area was covered by cells) were washed with PBS and separated into single cells by use of TrypLE Select (trade name, manufactured by Life Technologies). Then, the separated human ES single cells were seeded in plastic culture dishes coated with Laminin 511-E8 and cultured under feeder-free conditions in StemFit medium in the presence of Y-27632 (ROCK inhibitor, 10 μM). When 6-well plates (for cell culture, culture area: 9.4 cm², manufactured by AGC TECHNO GLASS., LTD) were used as the plastic culture dishes, the number of separated human ES single cells to be seeded was specified as 0.4 to 1.2×10⁴ cells per well. One day after seeding, the medium was exchanged with StemFit medium not containing Y-27632. Thereafter, the medium was exchanged with Y-27632-free StemFit medium once every 1 to 2 days. Thereafter, the cells were cultured under feeder-free conditions until 1 day before reaching sub-confluency. The human ES cells the 1 day before the sub-confluency were cultured for 1 day (preconditioning treatment) under feeder-free conditions in the presence of SB431542 (TGFβ signaling pathway inhibitor, 5 μM) and SAG (Shh signaling pathway agonist, 300 nM).

The human ES cells were washed with PBS, then treated for cell dispersions using TrypLE Select, and further separated into single cells by pipetting. Thereafter, the separated human ES single cells were suspended in 100 μL of a serum-free medium such that the density of cells per well of a non-cell adhesive 96-well culture plate (trade name: PrimeSurface, 96-well V-bottom plate, manufactured by Sumitomo Bakelite Co., Ltd.) was 1.2×10⁴ cells, and subjected to suspension culture in the conditions of 37° C.and 5% CO₂. The serum-free medium (gfCDM+KSR) used herein is a serum-free medium prepared by adding 10% KSR and 450 μM 1-monothioglycerol and 1× Chemically defined lipid concentrate to a mixture of culture fluids containing F-12 medium and IMDM medium in a ratio of 1:1.

At the initiation time of the suspension culture (Day 0 from initiation of the suspension culture), Y-27632 (ROCK inhibitor, final concentration: 10 μM) were added to the serum-free medium. On Day 3 from initiation of the suspension culture, 50 L of a culture medium not containing Y-27632 and containing human recombinant BMP4 (trade name: Recombinant Human BMP-4, manufactured by R&D) was added in such a manner that the final concentration of the exogeneous human recombinant BMP4 reached 0.15 nM or 1.5 nM. In addition, PD407824 (Tocris Bioscience) was added simultaneously with BMP4 at a final concentration of 1 μM. Day 6 or later from initiation of the suspension culture, a half of the medium was exchanged with a culture medium, which did not contain any of Y27632, human recombinant BMP4, and PD407824, once every 3 days.

Further, the aggregated masses (cell aggregates) of Day 14 from initiation of the suspension culture were transferred to a 90-mm low adhesive plate (manufactured by Sumitomo Bakelite Co., Ltd.) and cultured for 3 days in the conditions of 37° C. and 5% CO₂ in a serum-free medium (DMEM/F12 medium supplemented with 1% N2 Supplement) containing a Wnt signaling pathway agonist (CHIR99021, 3 M) and a FGF signaling pathway inhibitor (SU5402, 5 μM). The aforementioned method was performed also in subsequent Examples in the same manner, unless otherwise specified.

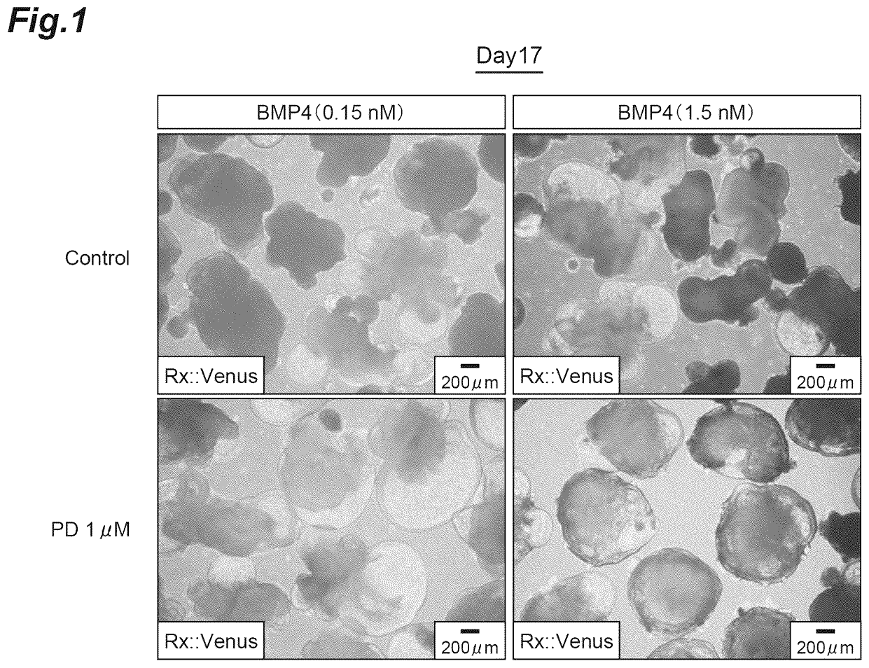

After culturing for 17 days from initiation of the suspension culture, the aggregated masses were observed. From the result, it was found that retinal tissue can be produced by using PD407824 (“PD” in the figure) in combination even if the amount of BMP added is decreased to about 1/10 of that in normal cases (0.15 nM). In addition, it was found that, through the use of PD407824 in combination, higher production efficiency for Rx::Venus-positive NR was obtained when 0.15 nM BMP4 was added than when 1.5 nM BMP4 was added. Furthermore, it was observed that, when PD407824 was added to 1.5 nM BMP4, a sphere-like cell aggregate comprising a multilayer structure in which a layer of retinal pigment epithelial (RPE) cells was localized in the outside and a layer of neural retinal tissue (NR) was localized in the inside was produced (FIG. 1).

Thus, it was found that the effect and efficiency of differentiation are enhanced by adding PD407824 in addition to BMP4, and that particularly enhanced effect and efficiency of differentiation are achieved with a combination of BMP4 at a low concentration and PD407824. In addition, it was found that, when BMP4 is combined with PD407824, RPE and NR are simultaneously produced by increasing the concentration of BMP4.

### Example 2 Confirmation of Promotion of Differentiation Into Rx::Venus-Positive Retina

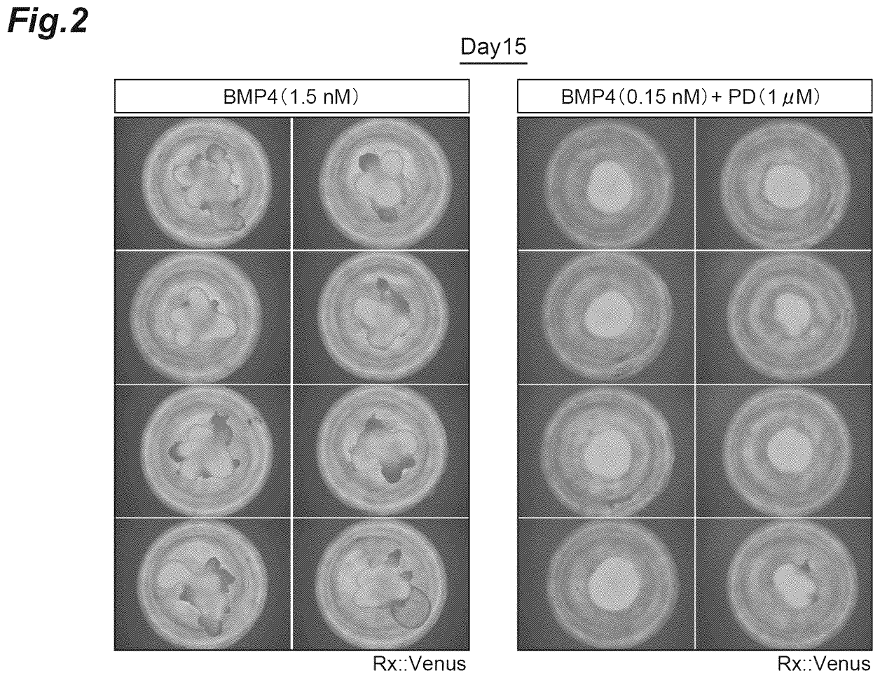

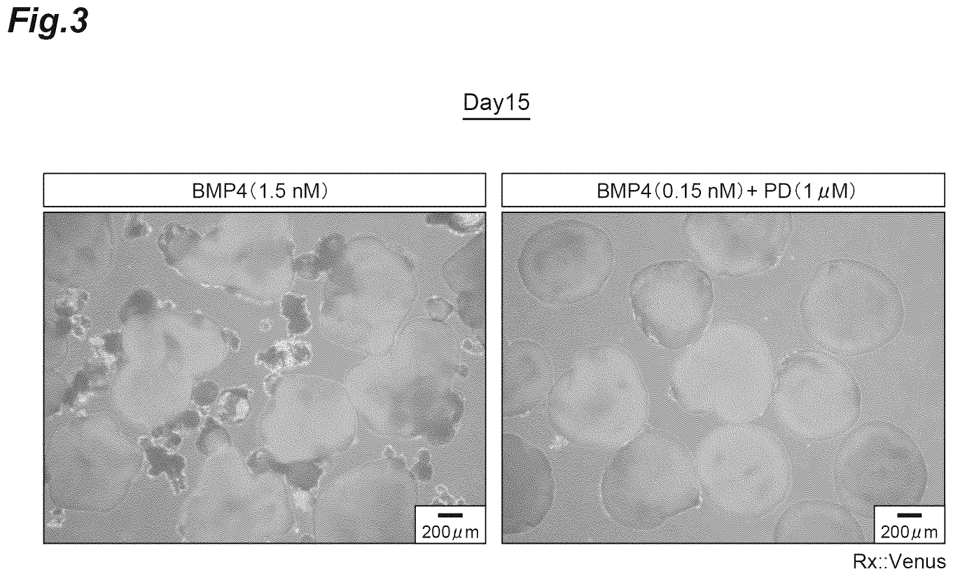

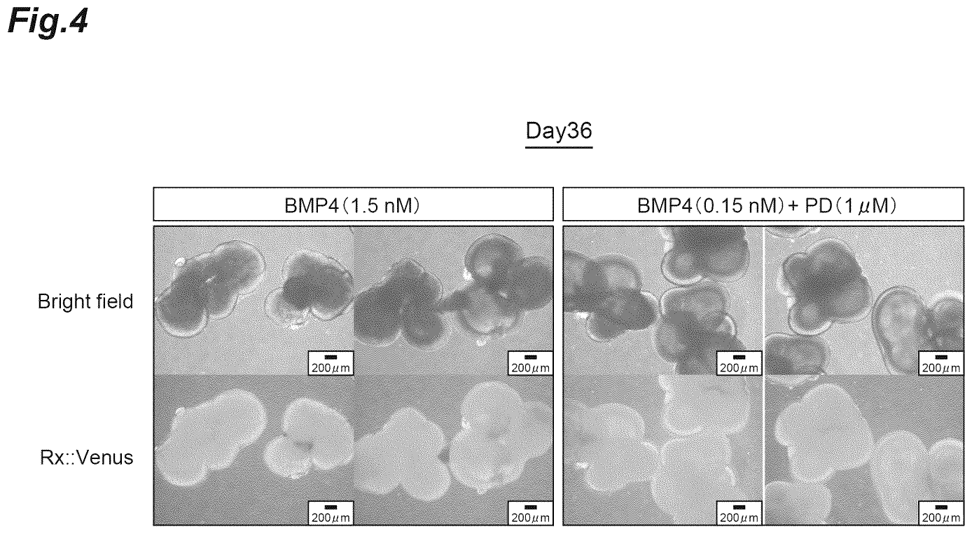

Aggregated masses produced as in Example 1 with addition of (1) 1.5 nM BMP4 alone, or (2) 1 μM PD407824 in addition to 0.15 nM BMP4 on Day 3 from initiation of the suspension culture were cultured for 15 days or 36 days from initiation of the suspension culture, and the aggregated masses were observed. From the result, it was found that a mass of Rx::Venus-negative cells attached to an Rx::Venus-positive aggregated mass to result in inclusion of non-target cells when 1.5 nM BMP4 was added, whereas Rx::Venus-negative masses were significantly reduced to result in high efficiency of differentiation into Rx::Venus-positive NR when PD407824 was added to 0.15 nM BMP4 (FIGS. 2 to 4). In addition, it was found that an aggregated mass (retinal tissue) of favorable shape (almost truly spherical shape) compared with that in the case of using BMP alone can be produced by combining BMP and PD407824. Specifically, when BMP was used alone, among 96 wells, a cell aggregate having an almost truly spherical shape was found in five wells, and a cloverleaf cell aggregate was found in 91 wells, whereas, when PD407824 was used in combination, among 96 wells, a cell aggregate having an almost truly spherical shape was found in 95 wells, and a cell aggregate with failure in aggregated mass formation was found in one well. Accordingly, almost 100% (about 99% (95/96), 100% if the case with failure in aggregated mass formation was excluded) of the cell aggregates produced were cell aggregates having an almost truly spherical shape.

### Example 3 Method for Differentiating Into Neural Retinal Tissue in Sphere-Like Cell Aggregate of Retinal Pigment Epithelial Cells

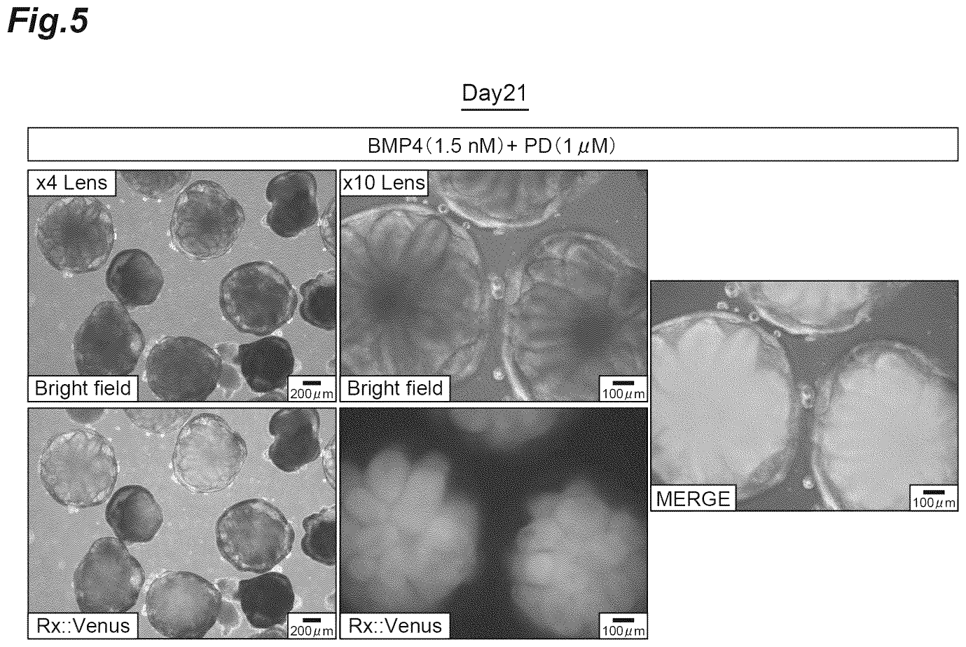

Aggregated masses produced as in Example 1 with addition of 1 μM PD407824 in addition to 1.5 nM BMP4 on Day 3 from initiation of the suspension culture were cultured for 21 days from initiation of the suspension culture and observed. As a result, it was observed that a sphere-like cell aggregate comprising a multilayer structure in which a layer of retinal pigment epithelial (RPE) cells was localized in the outside and a layer of neural retinal tissue (NR) was localized in the inside was produced. In addition, it was found that an aggregated mass (retinal tissue) of favorable shape (a shape with a plurality of overlapping true spheres (e.g., cloverleaf)) can be produced (FIG. 5).

The aggregated masses on Day 25 from initiation of the suspension culture were fixed with 4% PFA, subjected to replacement with 20% sucrose, and then frozen and sectioned. These frozen sections were subjected to immunostaining with DAPI, an anti-CHX10 antibody (trade name: Anti CHX10 Antibody, manufactured by EX alpha), an anti-MITF antibody (manufactured by EX alpha), an anti-collagen IV (manufactured by Abcam plc.) and an anti-Zo-1 antibody (Invitrogen). As necessary, antigen activation treatment (manufactured by Thermo Fisher Scientific) was performed with a microwave.

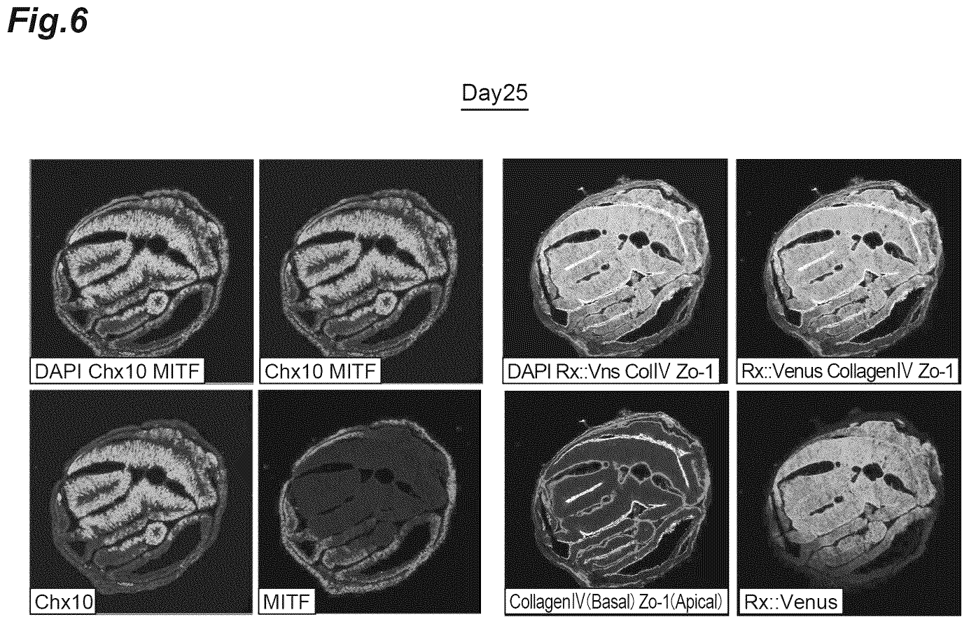

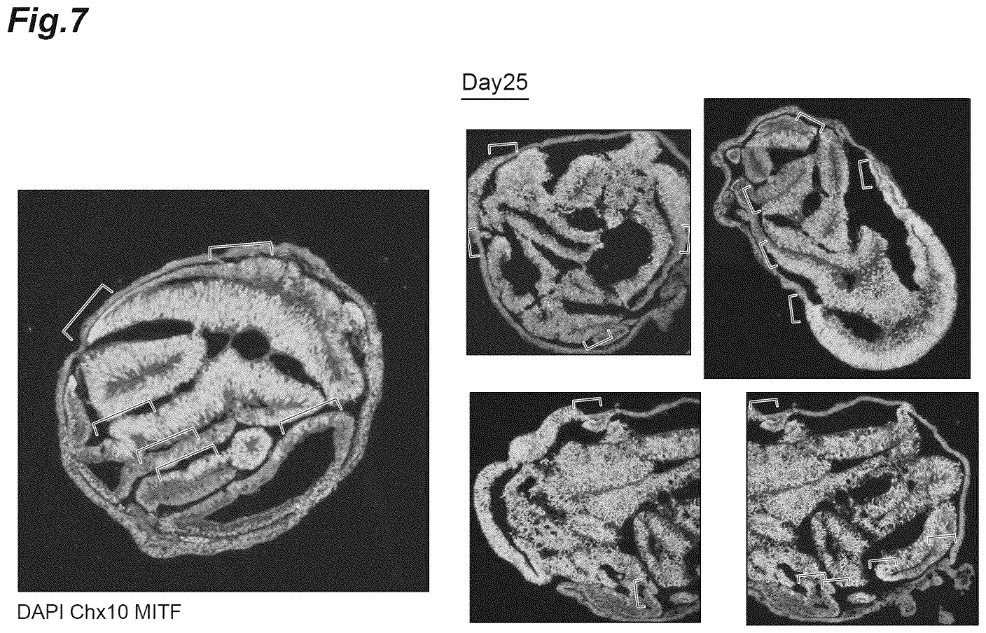

These immunostained sections were observed with a confocal fluorescence microscope (SP-8 manufactured by Leica). From the result, it was confirmed that a structure of a plurality of layers was formed in each aggregated mass, MITF-positive RPE cells were contained in the outermost cell layer of each aggregated mass, and Chx 10-positive neural retinal progenitor cells were contained in the inner cell layer. In addition, in the outer layer of RPE cells, a Zo-1-positive apical surface was formed in the outermost surface, and a collagen-positive basal surface was formed in the inside thereof. Furthermore, it was confirmed that, also in the inner neural retinal layer, a collagen-positive basal surface was formed in the outside and a Zo-1-positive apical surface was formed in the inside, and that an aggregated mass with apical-and-basal polarity was formed (FIG. 6). Moreover, many characteristic ciliary marginal zone (CMZ) structures (places indicated by “[” in FIG. 7, about 3 to 10 per aggregate) in a boundary part between the layer of MITF-positive RPE and the layer of a Chx10-positive neural retina were found through observation (FIG. 7).

Thus, it was found that neural retinal tissue including many CMZs and having polarity on the inner surface in contact with the outer layer of RPE cells in a sphere-like cell aggregate can be produced by adding PD407824 in addition to BMP4.

### Example 4 Examination of Concentrations of BMP4 and PD

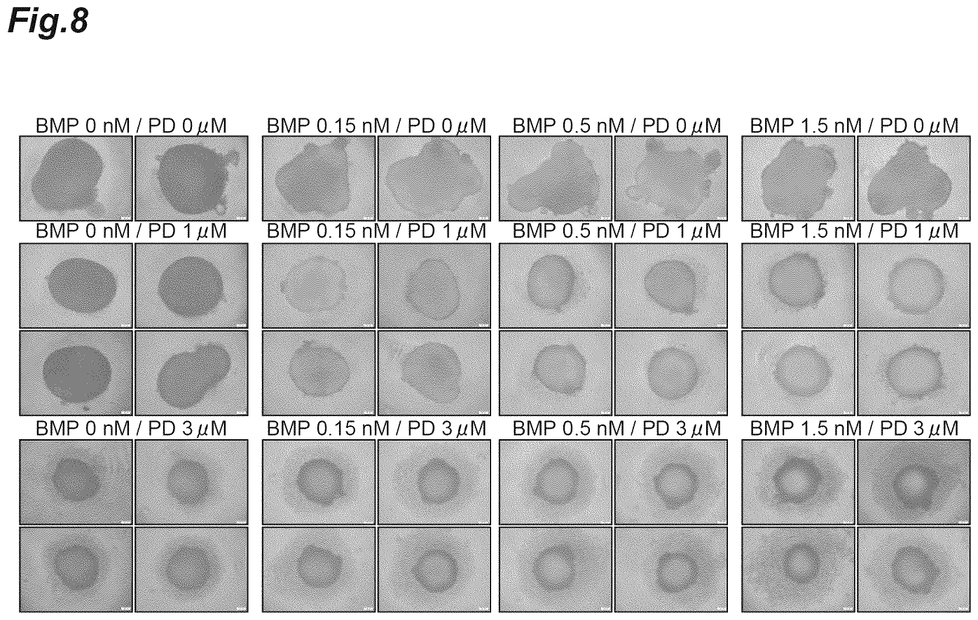

Aggregated masses produced as in Example 1 with addition of 1 μM or 3 μM PD407824 in addition to 0.15 nM, 0.5 nM or 1.5 nM on Day 3 from initiation of the suspension culture were cultured for 9 days from initiation of the suspension culture, and the aggregated masses were observed. From the result, it was found that an Rx::Venus-positive neural retina was formed through differentiation in the whole region of an aggregated mass when 1 μM PD was added in addition to 0.15 nM BMP4. On the other hand, it was confirmed that a sphere-like cell aggregate comprising a multilayer structure of an outer layer of RPE cells and an inner layer of neural retinal tissue including many CMZs and having polarity was produced when 1 μM or 3 μM PD407824 was added in addition to 0.5 nM or 1.5 nM BMP4 (FIG. 8).

Thus, it was found that a neural retinal tissue can be produced by adding 1 μM to 3 μM PD407824 in addition to 0.15 nM to 1.5 nM BMP4.

### Example 5 Confirmation of Differentiation Into Photoreceptor Cells Through Long-Term Culture>

Aggregated masses produced as in Example 1 by culturing under conditions involving addition of 1 μM PD407824 in addition to 1.5 nM BMP4 for 60 days from initiation of the suspension culture were fixed with 4% PFA, subjected to replacement with 20% sucrose, and then frozen and sectioned. These frozen sections were subjected to antigen activation treatment with a microwave, followed by immunostaining with DAPI, an anti-CHX10 antibody (trade name: Anti CHX10 Antibody, manufactured by EX alpha) and an anti-CRX antibody (manufactured by Takara Bio Inc.).

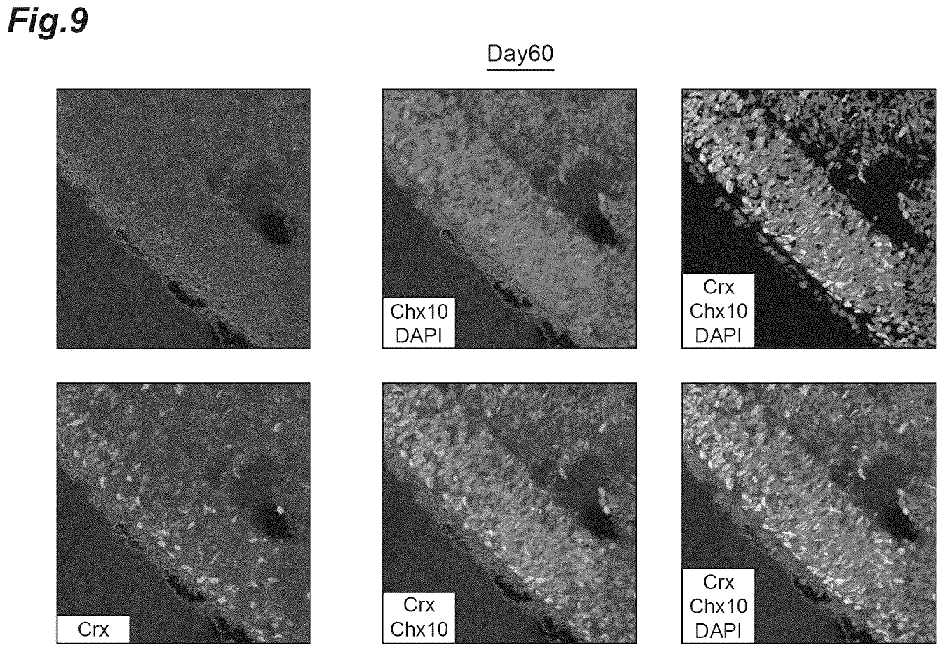

These immunostained sections were observed with a confocal fluorescence microscope (SP-8 manufactured by Leica). From the result, it was confirmed that RPE cells having black pigments were contained in the outermost cell layer of an aggregated mass and Chx10-positive retinal progenitor cells inside of which a layer structure was formed were contained. In addition, it was confirmed that Crx-positive photoreceptor cells were localized in the side close to RPE cells in the inner layer structure (FIG. 9).

Thus, it was found that an aggregated mass produced with addition of PD407824 to BMP4 differentiates into photoreceptor cells.

1. A method for producing a retinal cell or retinal tissue, comprising:
(A) suspension-culturing a pluripotent stem cell to form a cell aggregate of pluripotent stem cells; and
(B) suspension-culturing the cell aggregate obtained in the step (A) in the presence of a BMP signaling pathway agonist and a CHK1 signaling pathway inhibitor to obtain a cell aggregate containing a retinal cell.

2. The production method according to claim 1, wherein, in the step (B), the BMP signaling pathway agonist is present in such a concentration that an almost truly spherical cell aggregate is formed and differentiation into a retinal pigment epithelial cell is suppressed.

3. The production method according to claim 1, wherein the cell aggregate obtained in the step (B) is a sphere-like cell aggregate comprising a multilayer structure of an outer structure including a retinal pigment epithelial cell and an inner structure including a neural retina, and, in the step (B), the BMP signaling pathway agonist is present in such a concentration that the sphere-like cell aggregate comprising a multilayer structure is formed.

4. The production method according to claim 1, wherein the CHK1 signaling pathway inhibitor is PD407824.

5. The method according to claim 1, wherein the BMP signaling pathway agonist is one or more proteins selected from the group consisting of BMP2, BMP4, BMP7 and GDF7.

6. The production method according to claim 1, wherein, in the step (B), the BMP signaling pathway agonist is added to a culture medium between Day 2 and Day 9 after start of suspension culture in the step (A).

7. The production method according to claim 1, wherein, in the step (B), the CHK1 signaling pathway inhibitor is added to a culture medium simultaneously with the BMP signaling pathway agonist.

8. The production method according to claim 1, wherein a concentration of the CHK1 signaling pathway inhibitor is such a concentration that a CHK1 signaling pathway inhibitory effect comparable to a CHK1 signaling pathway inhibitory effect of 0.1 UM to 10 UM PD407824 is exerted.

9. The production method according to claim 1, further comprising dissecting retinal tissue of size required for transplantation from the cell aggregate obtained in the step (B).

10. A sphere-like cell aggregate comprising:
a multilayer structure of an outer structure including a retinal pigment epithelial cell and an inner structure including a neural retina, wherein
(1) a neural retinal layer including at least a photoreceptor layer is formed in the neural retina in the inner structure, and the photoreceptor layer includes one or more cells selected from the group consisting of a photoreceptor cell, a photoreceptor progenitor cell and a retinal progenitor cell,
(2) the neural retina is present in a folded state in the inner structure,
(3) the retinal pigment epithelial cell in the outer structure is an RPE65-positive cell, an MITF-positive cell, or an RPE65-positive and MITF-positive cell, and
(4) the cell aggregate is free of a lens, a vitreous body, a cornea and a blood vessel.

11. The sphere-like cell aggregate according to claim 10, wherein, in at least a part of the sphere-like cell aggregate, a basal surface of the retinal pigment epithelial cell is facing the inner structure, and a basal surface of the neural retina is facing the outer structure.

12. The sphere-like cell aggregate according to claim 10, wherein the retinal pigment epithelial cell and the neural retina are further connecting to each other as an epithelium structure, and the sphere-like cell aggregate further includes a ciliary marginal zone-like structure between the retinal pigment epithelial cell and the neural retina.

13. The sphere-like cell aggregate according to claim 12, wherein the ciliary marginal zone-like structure includes an Rdh10-positive cell, an Otx1-positive cell, and/or a Zic1-positive cell.

14. The sphere-like cell aggregate according to claim 10, wherein 30% or more of the inner structure constitutes a neural retina.

15. The sphere-like cell aggregate according to claim 10, having a diameter of 0.2 mm to 2 mm.

16. A method for producing the sphere-like cell aggregate according to claim 10, comprising:
(A) suspension-culturing a pluripotent stem cell to form a cell aggregate of pluripotent stem cells; and
(B) suspension-culturing the cell aggregate obtained in the step (A) in the presence of a BMP signaling pathway agonist and a CHK1 signaling pathway inhibitor to obtain a sphere-like cell aggregate comprising a multilayer structure of an outer structure including a retinal pigment epithelial cell and an inner structure including a neural retina.

17. A pharmaceutical composition comprising:
the sphere-like cell aggregate according to claim 10 or a part thereof.

18. A method for treating a disease caused by damage of a retinal cell or retinal tissue or injury of retinal tissue, comprising:
transplanting the sphere-like cell aggregate according to claim 10 or a part thereof to a subject in need of transplantation.
